(19) United States
(12) Patent Application Publication
(10) Pub. No.: US 2010/0041920 A1
(43) Pub. Date: Feb. 18, 2010

(54) NEW SALT FORMS OF AN AMINOINDAN DERIVATIVE

(76) Inventors: Stephen Benedict David Winter, Santa Coloma De Cervello (ES); Ernesto Duran Lopez, Castellbisal (ES); Monica Benito Velez, L'Hospitalet de Llobregat (ES)

Correspondence Address: IP Patent Docketing;K&L GATES LLP, 599 Lexington Avenue, 33rd Floor, New York, NY, 10022-6030, US

(21) Appl. No.: 12/505,612

(22) Filed: Jul. 20, 2009

Related U.S. Application Data
(60) Provisional application No. 61/082,057, filed on Jul. 18, 2008.

(51) Int. Cl. C07C 211/38; C07C 209/44

(52) U.S. Cl. 564/394; 564/428

(57) ABSTRACT

The present invention relates generally to novel salt forms of R-(+)-N-propargyl-1-aminoindan (i.e. rasagiline base), to a compound of formula Ia, to processes for their preparation and isolation, and to pharmaceutical compositions comprising the same.

### CROSS REFERENCE TO RELATED APPLICATIONS

This application claims priority to U.S. Provisional Application No. 61/082,057 filed Jul. 18, 2008, which is expressly incorporated herein by reference in its entirety.

### BACKGROUND

1. Field of the Invention

The present invention relates generally to novel salt forms of R-(+)-N-propargyl-1-aminoindan (i.e. rasagiline base), to a compound of formula Ia, to processes for their preparation and isolation, and to pharmaceutical compositions comprising the same.

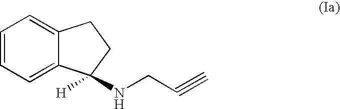

2. Relevant Background

Rasagiline mesylate is an active pharmaceutical substance with an empirical formula of C₁₂H₁₃N.CH₄O₃S and a molecular weight of 267.34. Rasagiline mesylate is the international common accepted name for R-(+)-N-propargyl-1-aminoindan mesylate (or (1R)—N-prop-2-yn-1-ylindan-1-amine mesylate or (1R)-2,3-dihydro-N-2-propynyl-1H-inden-1-amine mesylate), which is represented in Formula I.

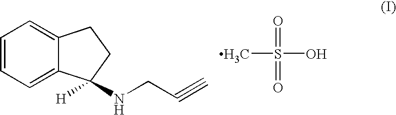

Rasagiline mesylate is a commercially marketed pharmaceutically active substance indicated for the treatment of the signs and symptoms of idiopathic Parkinson's disease as initial monotherapy and as adjunct therapy to levodopa. Rasagiline is a selective irreversible inhibitor of the B-form of monoamine oxidase enzyme (MAO-B). In the United States, rasagiline mesylate is marketed under the name Azilect® for the treatment of early Parkinson's disease.

Rasagiline base and its salts are claimed in EP Patent Application No. 0436492B1. In this reference, only rasagiline hydrochloride (m.p.=179-181° C., see example 3; m.p.=183-185° C., see example 4) and di-rasagiline L-tartrate (m.p.=175-177° C., see example 6) are prepared. Aside from the melting point of these salts, no further polymorphic characterization data is provided.

Rasagiline mesylate is specifically claimed in EP Patent Application No. 0812190B1. More precisely, in Example 6B of that patent, the product is obtained by treating the enantiopure rasagiline L-tartrate salt with methanesulfonic acid in isopropanol at reflux temperature for 30 minutes, allowing the reaction to cool to room temperature, and filtering the resulting precipitate. In this reference, other additional rasagiline salts are disclosed, i.e. rasagiline salts of tartrate (m.p.=176.2-177.3° C.), maleate (m.p.=87.2-87.8° C.), sulphate (m.p.=159.4-161.1° C.), hydrochloride (m.p.=177.0-180.0° C.), tosylate (m.p.=129.3-129.9° C.), fumarate (m.p.=125.4-126.2° C.), phosphate (m.p. 109.5-110.4° C.), esylate (m.p. not available), tannate (m.p. not available) and acetate (m.p. 69.2-69.7° C.). Again, however, aside from the melting point of some of these salts, no further characterization data is provided.

EP Patent Application No. 1892233A1 relates to polymorphic forms of rasagiline oxalate and rasagiline edisylate.

Different salt forms of the same pharmaceutically active moiety differ in their physical properties such as melting point, solubility, chemical reactivity, etc. These properties may appreciably influence pharmaceutical properties such as dissolution rate and bioavailability.

In addition, polymorphism is very common among pharmaceutical substances. It is commonly defined as the ability of any substance to exist in two or more crystalline phases that have a different arrangement and/or conformation of the molecules in the crystal lattice. Different polymorphic forms of the same pharmaceutically active moiety also differ in their physical properties such as melting point, solubility, chemical reactivity, etc. These properties may appreciably influence pharmaceutical properties such as dissolution rate and bioavailability.

Therefore, it would be desirable to prepare and characterize new rasagiline salt forms. Further, it would be desirable to have reliable processes for producing these rasagiline salts forms. Additionally, the various rasagiline salt forms could be used to prepare improved pharmaceutical compositions.

### SUMMARY OF THE INVENTION

The present invention relates generally to novel salt forms of R-(+)-N-propargyl-1-aminoindan (i.e. rasagiline base), to a compound of formula Ia, to processes for their preparation and isolation, and to pharmaceutical compositions comprising the same.

### BRIEF DESCRIPTION OF THE DRAWINGS

FIG. 1 illustrates the X-ray powder diffraction pattern (XRD) of rasagiline benzoate Form I.

FIG. 2 illustrates the infra-red spectrum (IR) of rasagiline benzoate Form I.

FIG. 3 illustrates the X-ray powder diffraction pattern (XRD) of rasagiline galactarate Form I.

FIG. 4 illustrates the infra-red spectrum (IR) of rasagiline galactarate Form I.

FIG. 5 illustrates the X-ray powder diffraction pattern (XRD) of rasagiline gluconate amorphous form.

FIG. 6 illustrates the infra-red spectrum (IR) of rasagiline gluconate amorphous form.

FIG. 7 illustrates the X-ray powder diffraction pattern (XRD) of rasagiline glucuronate amorphous form.

FIG. 8 illustrates the infra-red spectrum (IR) of rasagiline glucuronate amorphous form.

FIG. 9 illustrates the X-ray powder diffraction pattern (XRD) of rasagiline tosylate Form I.

FIG. 10 illustrates the infra-red spectrum (IR) of rasagiline tosylate Form I.

FIG. 11 illustrates the X-ray powder diffraction pattern (XRD) of rasagiline phosphate amorphous form.

FIG. 12 illustrates the infra-red spectrum (IR) of rasagiline phosphate amorphous form.

FIG. 13 illustrates the X-ray powder diffraction pattern (XRD) of rasagiline maleate Form I.

FIG. 14 illustrates the infra-red spectrum (IR) of rasagiline maleate Form I.

FIG. 15 illustrates the X-ray powder diffraction pattern (XRD) of rasagiline succinate Form I.

FIG. 16 illustrates the infra-red spectrum (IR) of rasagiline succinate Form I.

FIG. 17 illustrates the X-ray powder diffraction pattern (XRD) of rasagiline acetate Form I.

FIG. 18 illustrates the infra-red spectrum (IR) of rasagiline acetate Form I.

FIG. 19 illustrates the X-ray powder diffraction pattern (XRD) of rasagiline acetate Form II.

FIG. 20 illustrates the infra-red spectrum (IR) of rasagiline acetate Form II.

FIG. 21 illustrates the X-ray powder diffraction pattern (XRD) of rasagiline tartrate Form I.

FIG. 22 illustrates the infra-red spectrum (IR) of rasagiline tartrate Form I.

FIG. 23 illustrates the X-ray powder diffraction pattern (XRD) of rasagiline hemitartrate Form I.

FIG. 24 illustrates the infra-red spectrum (IR) of rasagiline hemitartrate Form I.

FIG. 25 illustrates the X-ray powder diffraction pattern (XRD) of rasagiline fumarate Form I.

FIG. 26 illustrates the infra-red spectrum (IR) of rasagiline fumarate Form I.

FIG. 27 illustrates the X-ray powder diffraction pattern (XRD) of rasagiline hydrochloride Form I.

FIG. 28 illustrates the infra-red spectrum (IR) of rasagiline hydrochloride Form I.

FIG. 29 illustrates the X-ray powder diffraction pattern (XRD) of rasagiline hydrochloride Form II.

FIG. 30 illustrates the infra-red spectrum (IR) of rasagiline hydrochloride Form II.

FIG. 31 illustrates the X-ray powder diffraction pattern (XRD) of rasagiline besylate Form I.

FIG. 32 illustrates the infra-red spectrum (IR) of rasagiline besylate Form I.

FIG. 33 illustrates the simulated X-ray diffractogram (XR) for single crystal of rasagiline hydrochloride Form II.

FIG. 34 illustrates the molecular structure of rasagiline hydrochloride Form II with the atom-labeling scheme.

### DETAILED DESCRIPTION OF THE PREFERRED EMBODIMENTS

Reference will now be made in detail to the preferred embodiments of the invention. This invention may, however, be embodied in many different forms and should not be construed as limited to the embodiments set forth herein.

It will be apparent to those skilled in the art that various modifications and variations can be made in the present invention and specific examples provided herein without departing from the spirit or scope of the invention. Thus, it is intended that the present invention covers the modifications and variations of this invention that come within the scope of any claims and their equivalents.

The present invention relates generally to novel salt forms of R-(+)-N-propargyl-1-aminoindan (i.e. rasagiline base), to a compound of formula Ia, to processes for their preparation and isolation, and to pharmaceutical compositions comprising the same.

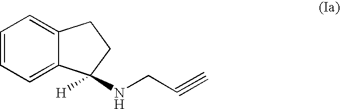

The novel salt forms of rasagiline have been prepared and structurally characterized as described herein and are referred to herein as rasagiline benzoate crystalline form (Form I), rasagiline galactarate crystalline form (Form I), rasagiline gluconate amorphous form, rasagiline D-glucuronate amorphous form, rasagiline tosylate crystalline form (Form I), rasagiline phosphate amorphous form, rasagiline maleate crystalline form (Form I), rasagiline succinate crystalline form (Form I), rasagiline acetate crystalline forms (Forms I and II), rasagiline L-tartrate salt crystalline form (Form I), rasagiline L-hemihydrate salt crystalline (Form I), rasagiline fumarate crystalline form (Form I), rasagiline hydrochloride crystalline forms (Forms I and II), and rasagiline besylate crystalline form (Form I).

The solid form salts of rasagiline of the present invention have been characterized by means of Powder X-ray diffraction pattern (XRD) and Fourier Transform Infrared (FTIR) spectra.

Surprisingly, a selected group of the novel salt forms of rasagiline of the present invention exhibit an excellent flowability, which might enhance their pharmaceutical properties as compared with the rasagiline mesylate salt that is currently marketed.

Flowability affects the ease with which the material is handled during processing into a pharmaceutical product. Namely, when flowability is very poor, problems occur with handling and processing during milling and formulating. The flowability of rasagiline salts can be measured using the Hausner ratio, which is a value calculated by dividing the tapped bulk density of the rasagiline salt by the freely settled bulk density of the rasagiline salt. The freely settled bulk density is calculated by pouring a known weight of material into a measuring cylinder and recording the volume. The tapped density is calculated by tapping the cylinder against a surface for a specified number of times and recording again the new volume. See Henry H. Hausner, “Friction Conditions in a Mass of Metal Powders,” Int. J. Powder Metall. Vol. 3, 1967, pp 7-13.

A low Hausner ratio indicates a high flowability. In this regard, it is generally accepted that a Hausner ratio equal to or higher than 1.46 indicates a very poor flowing material, which is rarely acceptable for manufacturing purposes. Therefore, a Hausner ratio less than 1.46 indicates an acceptable flowing material. Table 1 below summarizes the terms used to describe the flowability character with reference to the Hausner ratio value.

TABLE 1

| Flow Character | Hausner Ratio |
|---|---|
| Excellent | 1.00-1.11 |
| Good | 1.12-1.18 |
| Fair | 1.19-1.25 |
| Passable | 1.26-1.34 |
| Poor | 1.35-1.45 |
| Very poor | 1.46-1.59 |
| Very, very poor | >1.60 |

We have found that the rasagiline mesylate obtained by the processes disclosed in the prior art, which all make use of isopropanol as a crystallization solvent, shows poor flowability characteristics. Namely, the rasagiline mesylate obtained after a standard crystallization from isopropanol has a Hausner ratio equal to 1.72 (i.e. very, very poor flow character according to Table 1). Surprisingly, the selected group of rasagiline salts of the present invention, which comprises rasagiline succinate salt Form I, rasagiline L-hemitartrate salt Form I, rasagiline besylate salt Form I, and rasagiline hydrochloride salt Form II, have been found to show excellent flowability characteristics. Thus, the selected group of rasagiline salts of the present invention are better handled and processed during milling and formulating, as compared with the rasagiline mesylate salt that is currently marketed. Consequently, the selected salt forms of rasagiline of the invention are more suitable for pharmaceutical formulation use.

Further, the selected salt forms of rasagiline of the present invention exhibit a high solubility profile in water, i.e. higher than 50 mg/mL, and hence also show enhanced pharmaceutical properties regarding the dissolution rate and bioavailability.

In addition, the selected crystalline salt forms of rasagiline of the present invention have been found to be stable in terms of visual aspect, chemical purity and of polymorphic form either after 10 months of storage or after being submitted to accelerated stability conditions (40° C. and 75% RH) for one and/or two months, which also makes them suitable for pharmaceutical formulation use.

Additionally, the formation of the selected rasagiline salts of the invention might be an efficient way of purifying rasagiline base.

A first aspect of the present invention includes rasagiline benzoate salt and processes for obtaining it.

Another aspect of the present invention relates to a new rasagiline benzoate crystalline Form I and processes for obtaining it.

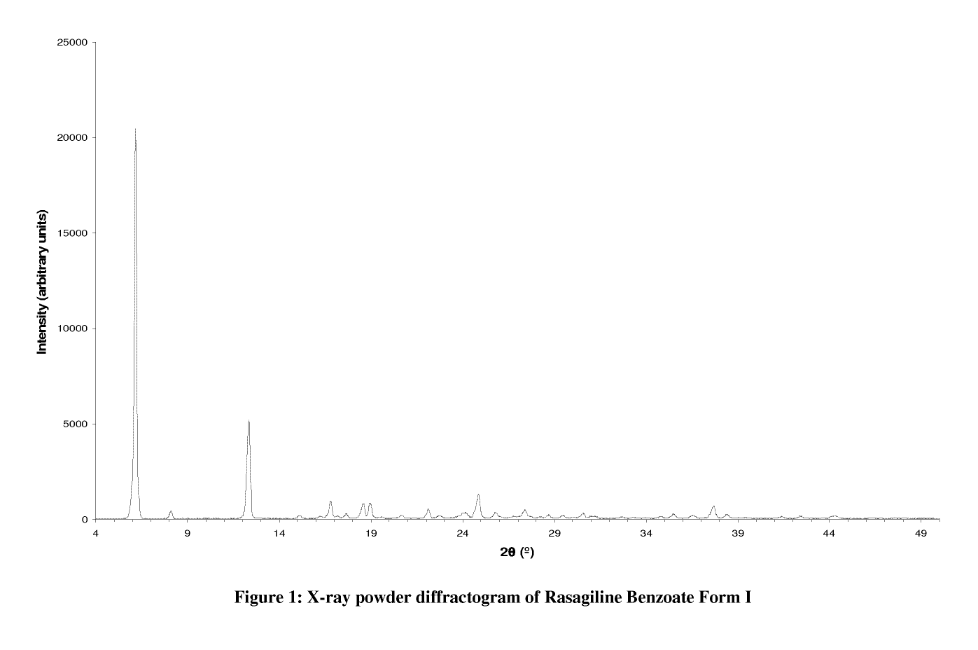

The rasagiline benzoate crystalline Form I of the present invention shows an XRD pattern (2θ) (±0.2°) having characteristic peaks at approximately 6.2, 8.1, 12.3, 16.8, 17.7, 18.6, 19.0, 22.1, 24.9, 27.4, 30.6, 37.7°. FIG. 1 illustrates the XRD of rasagiline benzoate crystalline Form I.

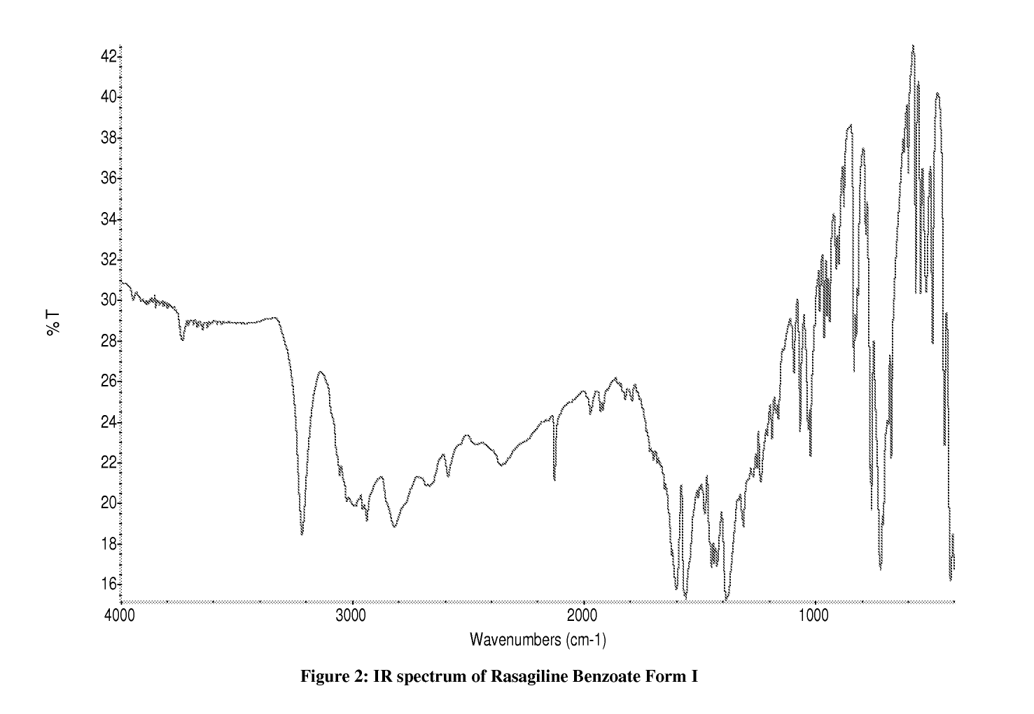

The rasagiline benzoate crystalline Form I of the present invention shows an IR spectrum having characteristic peaks at approximately: 3217.7, 2937.9, 2818.9, 2586.6, 2359.8, 2125.8, 1971.8, 1929.3, 1600.3, 1558.5, 1477.8, 1448.6, 1424.0, 1385.6, 1312.2, 1236.8, 1188.8, 1158.8, 1093.6, 1065.9, 1020.1, 833.2, 757.9, 717.6, 672.2, 442.2, 416.2 cm⁻¹ and with further peaks at: 980.7, 962.6, 949.3, 937.2, 910.9, 899.5, 824.7, 566.6, 546.0, 520.6, 494.6 cm⁻¹. FIG. 2 illustrates the IR spectrum of rasagiline benzoate crystalline Form I.

The rasagiline benzoate Form I of the present invention has been found to be highly stable in terms of polymorphic form after ten months of storage.

Another aspect of the invention relates to a process for preparing rasagiline benzoate salt form, said process comprising contacting rasagiline base with benzoic acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention includes rasagiline galactarate salt and processes for obtaining it.

Another aspect of the present invention relates to a new rasagiline galactarate crystalline Form I and processes for obtaining it.

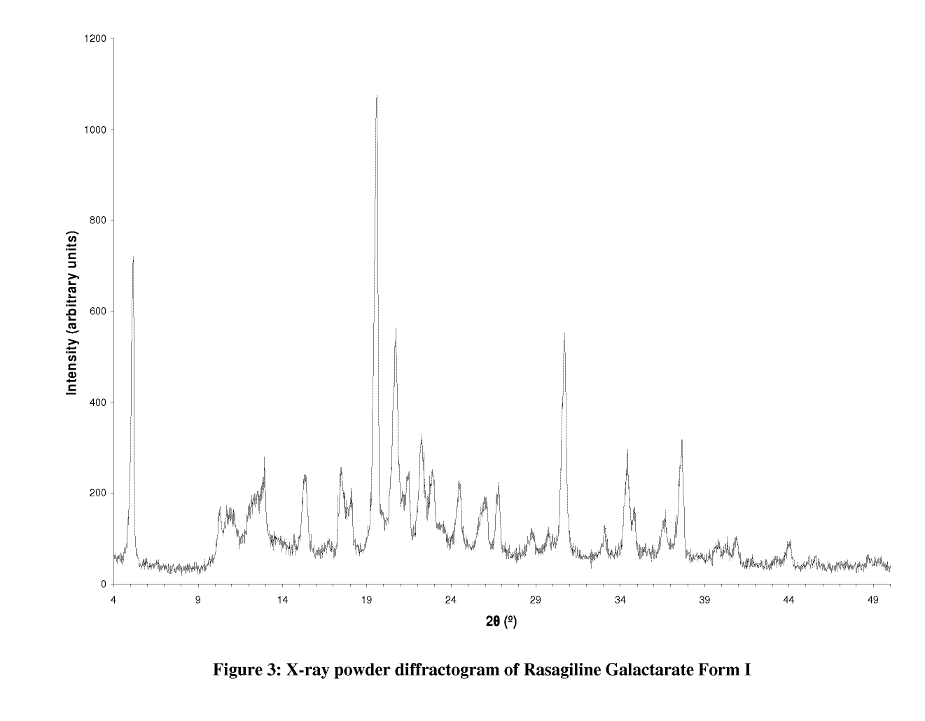

The rasagiline galactarate crystalline Form I of the present invention shows an XRD pattern (2θ) (±0.2°) having characteristic peaks at approximately: 5.1, 19.6, 20.7, 22.3, 30.7, 37.6° with further peaks at: 10.2, 10.9, 12.9, 15.3, 17.4, 18.1, 21.4, 22.9, 24.4, 25.9, 26.8, 34.4, 36.6°. FIG. 3 illustrates the XRD of rasagiline galactarate crystalline Form I.

The rasagiline galactarate crystalline Form I of the present invention shows an IR spectrum having characteristic peaks at approximately: 3286.2, 3220.3, 2935.4, 2806.3, 2586.2, 1721.6, 1593.9, 1478.8, 1434.9, 1368.0, 1314.9, 1238.0, 1109.2, 1049.3, 764.7, 666.8, 634.1, 509.0, 464.9, 444.0 cm⁻¹ with further peaks at: 2347.4, 2126.5, 968.0, 918.2, 862.4, 799.6 cm⁻¹.

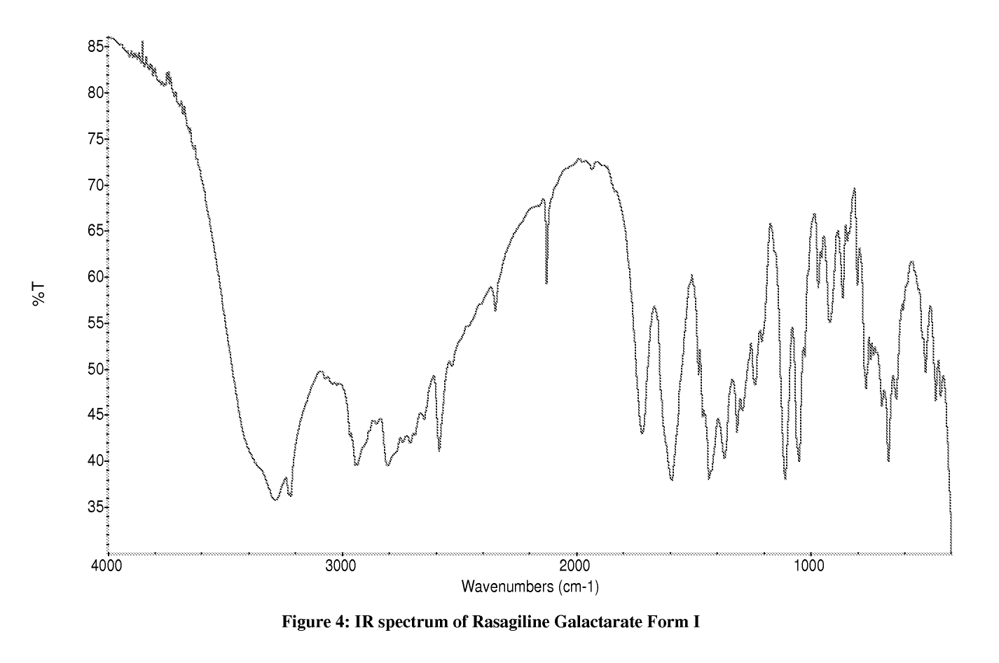

FIG. 4 illustrates the IR spectrum of rasagiline galactarate crystalline Form I.

The 1:1 salt correlation of rasagiline galactarate was confirmed by ¹H NMR spectrum.

The rasagiline galactarate Form I of the invention has a purity higher than about 98.8% relative peak area by HPLC. In addition, the rasagiline galactarate Form I of the present invention has been found to be stable in terms of chemical purity and of polymorphic form after ten months of storage.

Another aspect of the invention relates to a process for preparing rasagiline galactarate salt form, said process comprising contacting rasagiline base with galactaric acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention includes rasagiline gluconate salt and processes for its preparation and isolation.

Another aspect of the present invention relates to a new rasagiline gluconate amorphous form and processes for obtaining it.

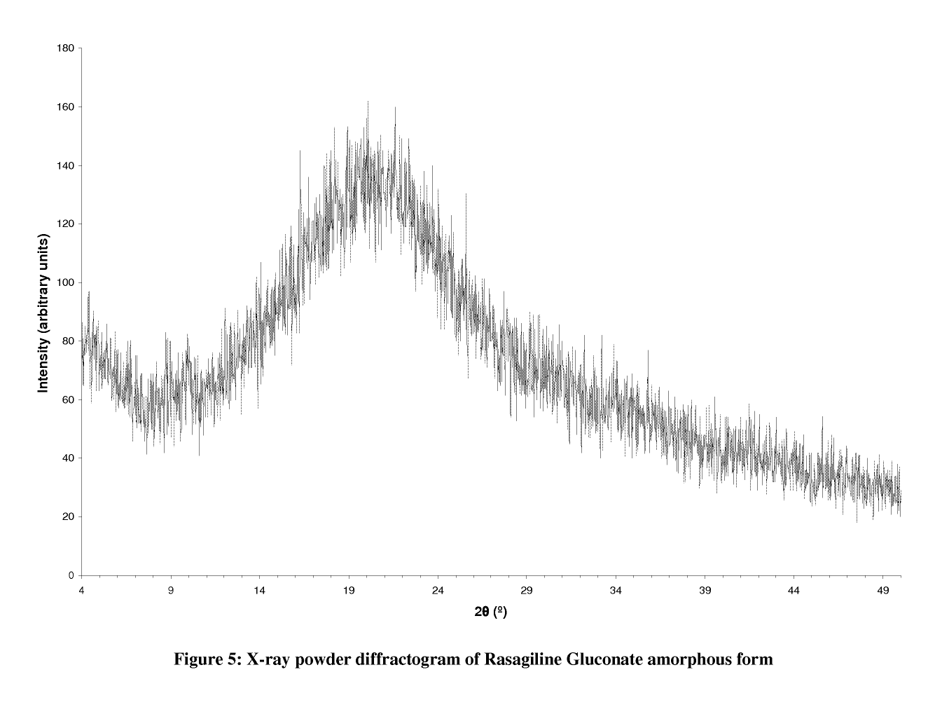

The rasagiline gluconate amorphous form of the present invention shows an X-ray diffraction pattern substantially as illustrated in FIG. 5.

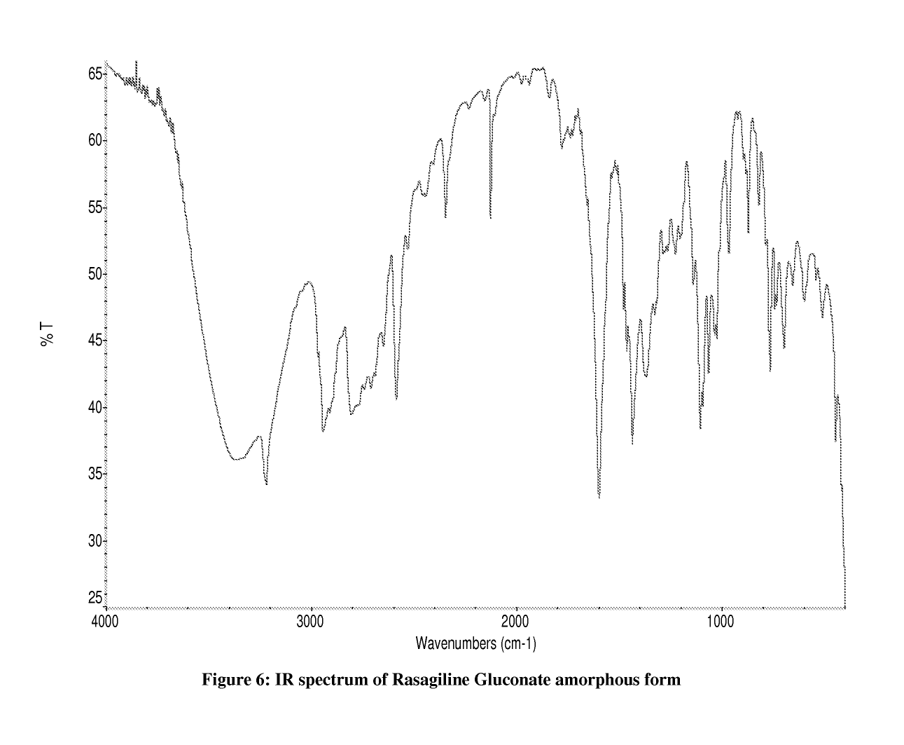

The rasagiline gluconate amorphous form of the present invention shows an IR spectrum having characteristic peaks at approximately: 3367.6, 3219.5, 2942.9, 2804.2, 2586.0, 1598.6, 1478.4, 1463.6, 1436.1, 1367.0, 1138.4, 1105.8, 1064.3, 1024.1, 765.1, 741.4, 696.2, 654.0, 597.1, 510.1, 444.8 cm⁻¹ with further peaks at: 2346.7, 2125.8, 1285.7, 1224.8, 966.6, 869.9, 820.5 cm⁻¹. FIG. 6 illustrates the IR spectrum of rasagiline gluconate amorphous form.

The 1:1 salt correlation of rasagiline gluconate was confirmed by ¹H NMR spectrum.

The rasagiline gluconate amorphous form of the invention has a purity higher than about 98.6% relative peak area by HPLC. In addition, the rasagiline gluconate amorphous form of the present invention has been found to be stable in terms of polymorphic form after ten months of storage.

Another aspect of the invention relates to a process for preparing rasagiline gluconate salt form, said process comprising contacting rasagiline base with gluconic acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention includes rasagiline D-glucuronate salt and processes for its preparation and isolation.

Another aspect of the present invention relates to a new rasagiline D-glucuronate amorphous form and processes for obtaining it.

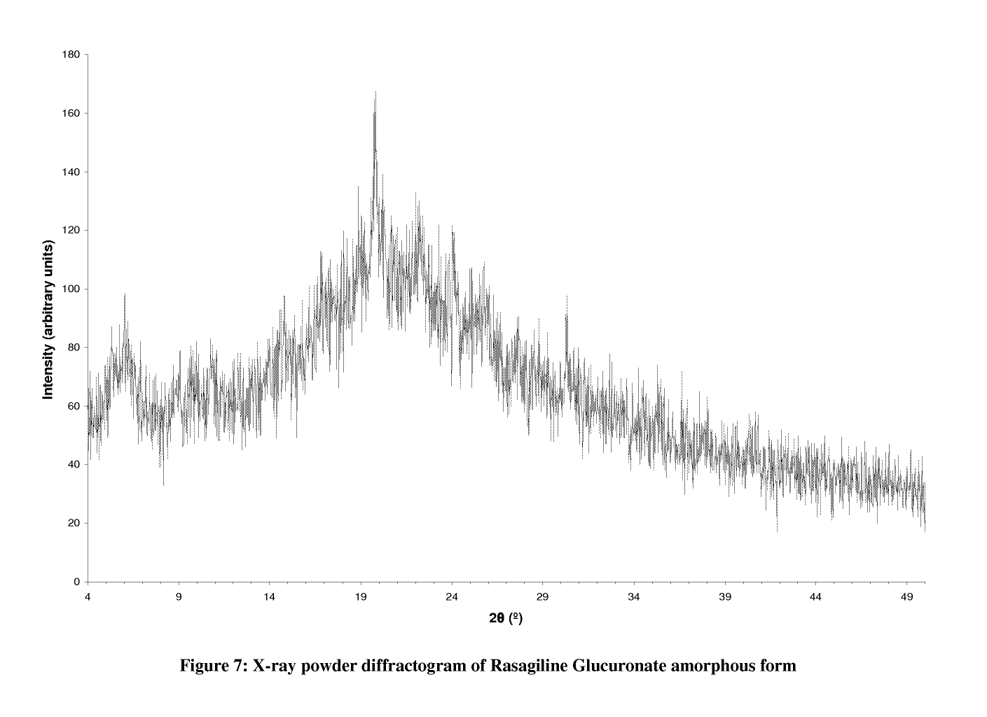

The rasagiline D-glucuronate amorphous form of the present invention shows an X-ray diffraction pattern substantially as illustrated in FIG. 7.

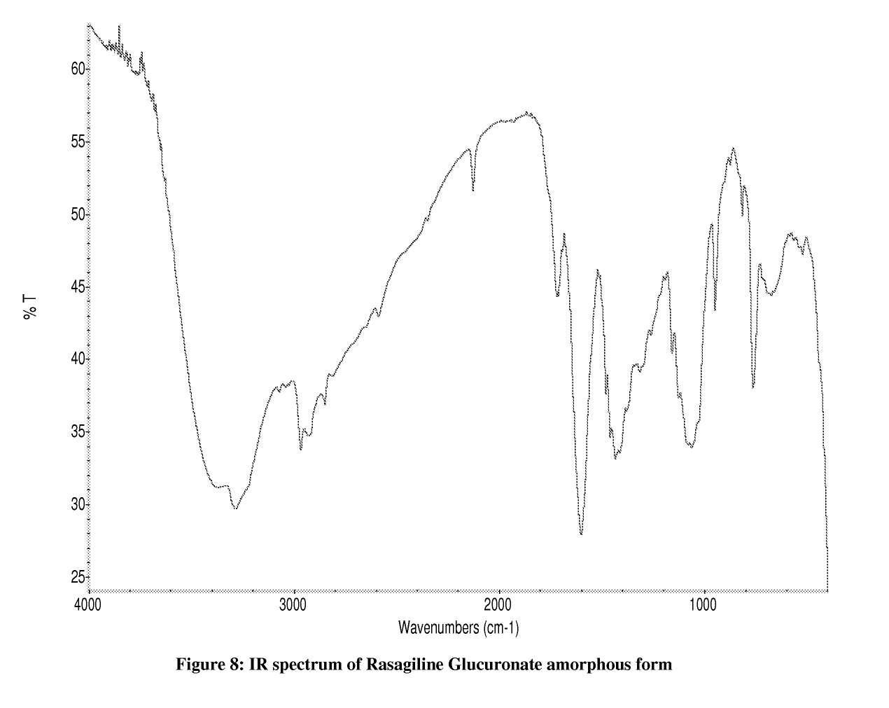

The rasagiline D-glucuronate amorphous form of the present invention shows an IR spectrum having characteristic peaks at approximately: 3285.9, 2968.2, 2127.2, 1720.6, 1600.9, 1479.8, 1433.7, 1156.9, 1063.1, 948.6, 816.3, 762.9, 674.0, and 520.7 cm⁻¹. FIG. 8 illustrates the IR spectrum of rasagiline glucuronate amorphous form.

The 1:1 salt correlation of rasagiline D-glucuronate was confirmed by ¹H NMR spectrum.

The rasagiline D-glucuronate amorphous form of the invention has a purity higher than about 86.0% relative peak area by HPLC. In addition, the rasagiline D-glucuronate amorphous form of the present invention has been found to be highly stable in terms of polymorphic form after ten months of storage.

Another aspect of the invention relates to a process for preparing rasagiline D-glucuronate salt form, said process comprising contacting rasagiline base with D-glucuronic acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention relates to a new rasagiline tosylate crystalline Form I and processes for obtaining it.

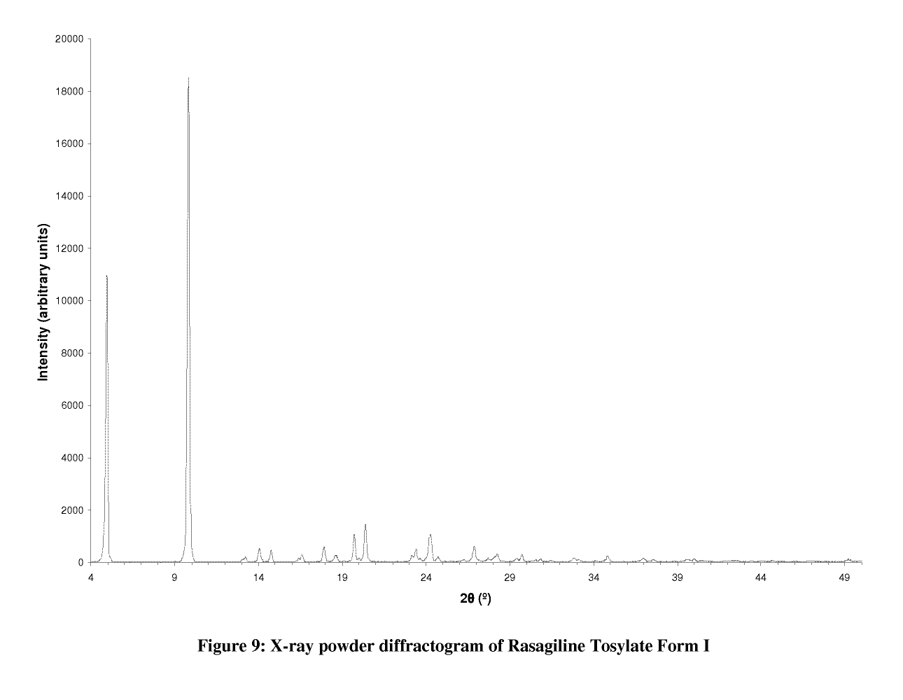

The rasagiline tosylate crystalline Form I of the present invention shows an XRD pattern (2θ) (±0.2°) having characteristic peaks at approximately: 4.9, 9.8, 19.7, 20.4, 24.3° with further peaks at: 14.1, 14.8, 16.6, 17.9, 18.6, 23.4, 26.9, 29.7°. FIG. 9 illustrates the XRD of rasagiline tosylate crystalline Form I.

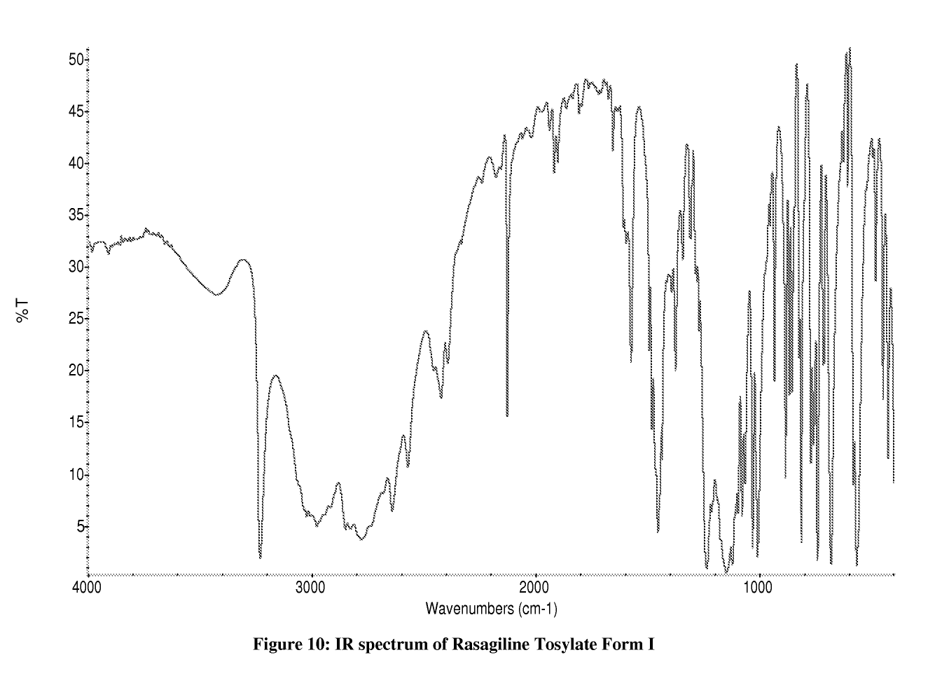

The rasagiline tosylate crystalline Form I of the present invention shows an IR spectrum having characteristic peaks at approximately: 3231.4, 2979.3, 2780.4, 2642.6, 2571.7, 2423.9, 2393.0, 2128.0, 1574.3, 1480.4, 1455.4, 1435.9, 1376.4, 1097.8, 1077.7, 1065.2, 1031.6, 1007.9, 933.2, 883.8, 866.9, 854.2, 813.5, 771.6, 758.8, 740.2, 716.8, 711.9, 681.5, 579.7, 565.4, 447.5, 426.2 cm⁻¹ with further peaks at: 3429.9, 1492.7, 1270.8, 1236.9, 1147.2, 823.3 cm⁻¹. FIG. 10 illustrates the IR spectrum of rasagiline tosylate crystalline Form I.

The rasagiline tosylate Form I of the invention has a purity higher than about 79.8% relative peak area by HPLC. In addition, the rasagiline tosylate Form I of the present invention has been found to be highly stable in terms of polymorphic form after ten months of storage.

Another aspect of the invention relates to a process for preparing rasagiline tosylate crystalline Form I, said process comprising contacting rasagiline base with p-toluene sulfonic acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention relates to a new rasagiline phosphate amorphous form and processes for obtaining it.

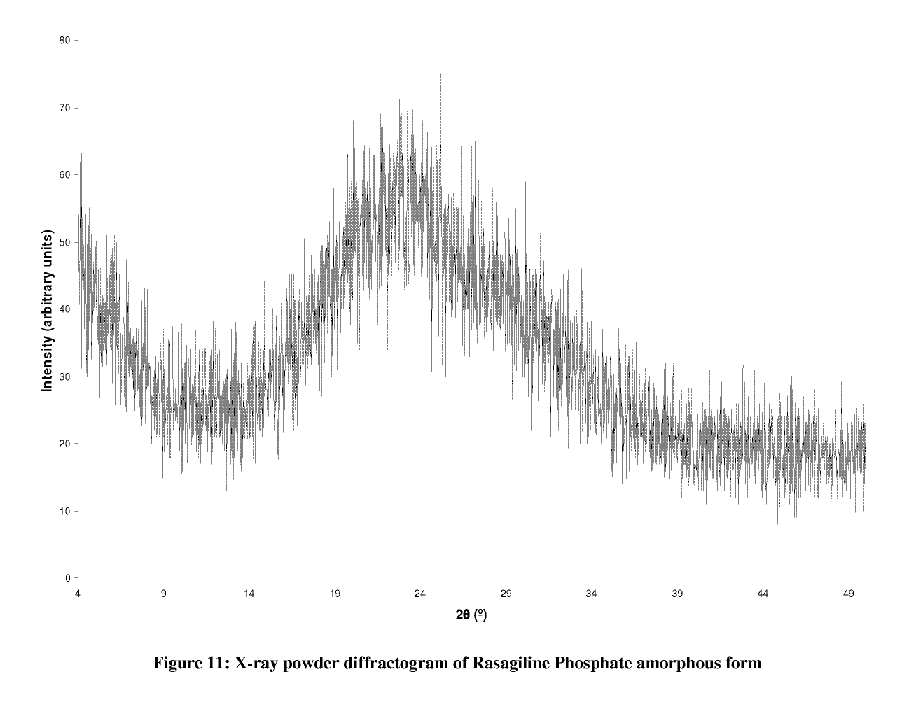

The rasagiline phosphate amorphous form of the present invention shows an X-ray diffraction pattern substantially as illustrated in FIG. 11.

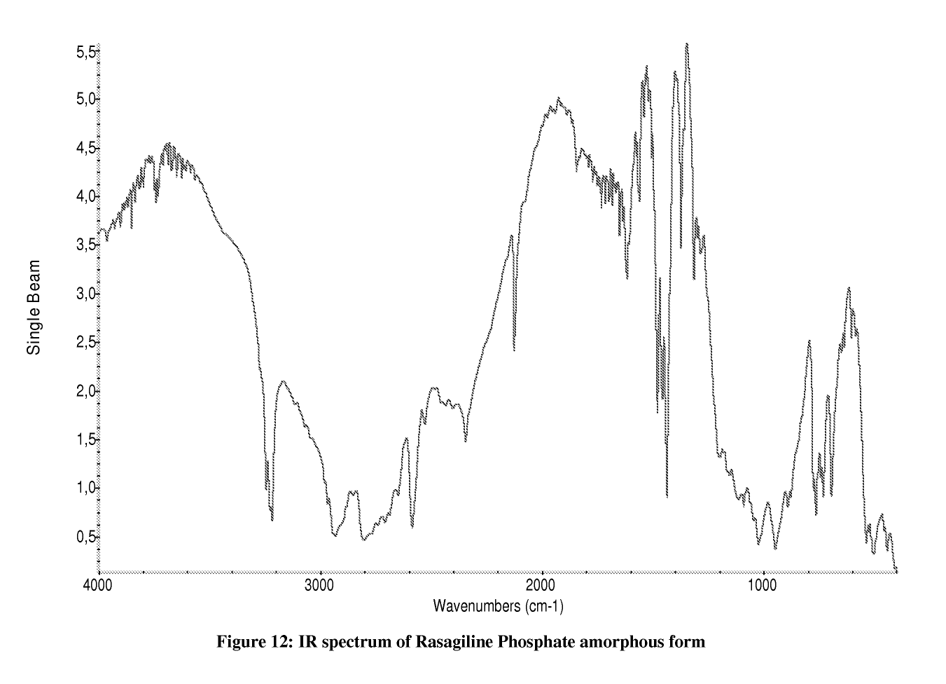

The rasagiline phosphate amorphous form of the present invention shows an IR spectrum having characteristic peaks at approximately: 3246.2, 3219.3, 2934.0, 2806.0, 2586.3, 2346.5, 2125.6, 1617.4, 1479.4, 1458.3, 1437.5, 1374.5, 1316.1, 1023.8, 947.0, 764.8, 731.5, 694.8, 604.1, 507.1 cm⁻¹. FIG. 12 illustrates the IR spectrum of rasagiline phosphate amorphous form.

The rasagiline phosphate amorphous form of the invention has a purity higher than about 98.5% relative peak area by HPLC.

Another aspect of the invention relates to a process for preparing rasagiline phosphate amorphous form, said process comprising contacting rasagiline base with phosphoric acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the present invention relates to a new rasagiline maleate crystalline Form I and processes for obtaining it.

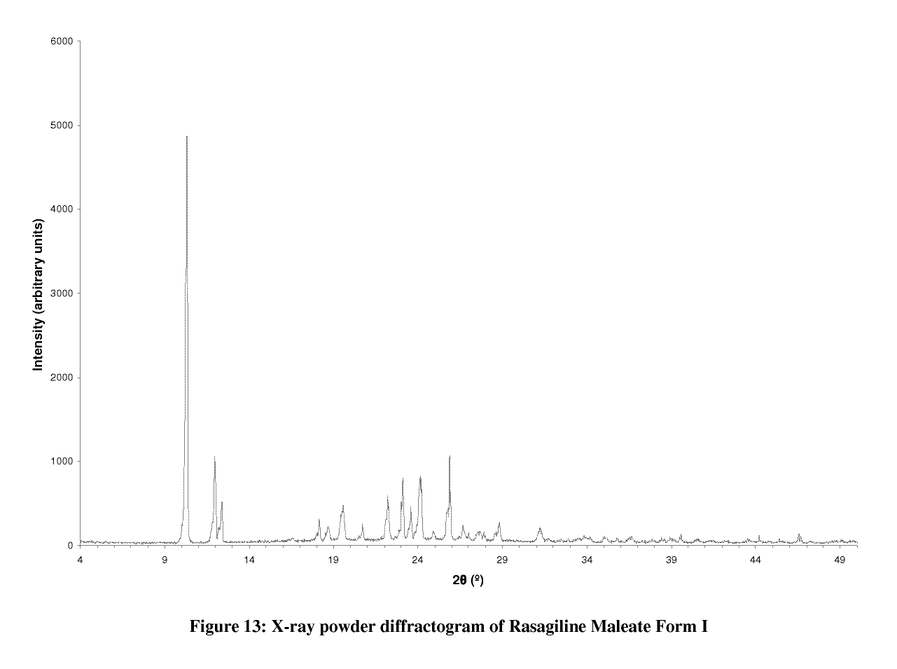

The rasagiline maleate crystalline Form I of the present invention shows an XRD pattern (2θ) (±0.2°) having characteristic peaks at approximately: 10.3, 12.0, 23.1, 24.1, 25.9° with further peaks at: 10.0, 12.4, 18.2, 18.7, 19.5, 20.7, 22.2, 23.6, 26.7, 28.8°. FIG. 13 illustrates the XRD of rasagiline maleate crystalline Form I.

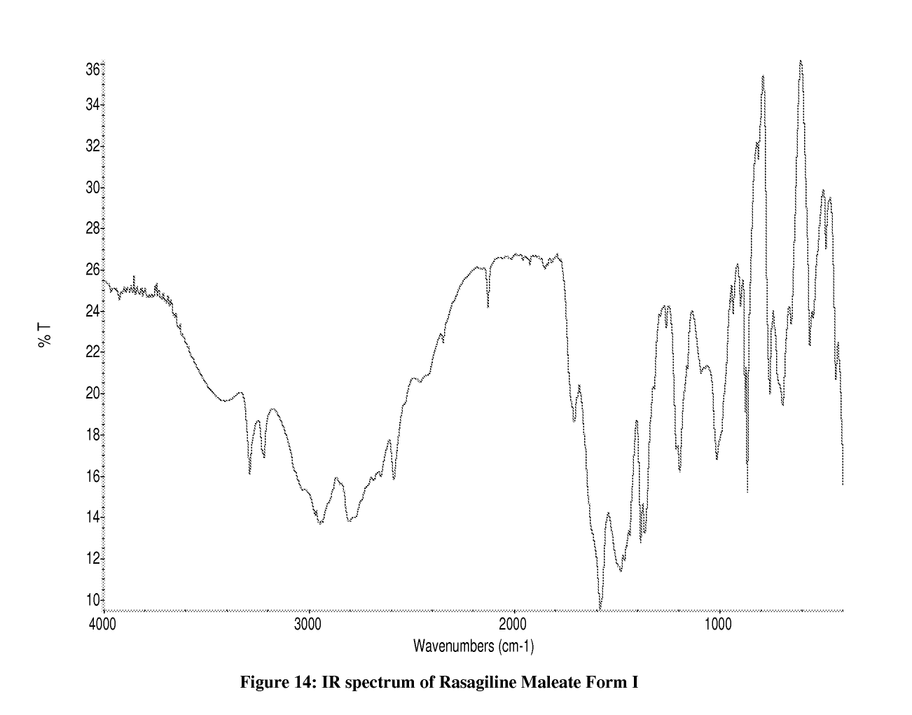

The rasagiline maleate crystalline Form I of the present invention shows an IR spectrum having characteristic peaks at approximately: 3288.8, 3219.4, 2945.3, 2802.4, 2586.7, 1583.2, 1480.1, 1384.8, 1364.2, 1193.9, 1012.9, 864.0 cm⁻¹ with further peaks at: 1708.3, 875.3, 756.8, 693.1, 434.0 cm⁻¹. FIG. 14 illustrates the IR spectrum of rasagiline maleate crystalline Form I.

The 1:1 salt correlation of rasagiline maleate was confirmed by ¹H NMR spectrum.

Another aspect of the invention relates to a process for preparing rasagiline maleate crystalline Form I, said process comprising contacting rasagiline base with maleic acid, optionally in the presence of a suitable solvent, and removing the solvent when necessary.

The suitable solvents comprise at least a C₁-C₅ alcohol solvent, water and mixtures thereof. The preferred C₁-C₅ alcohol solvent is 2-propanol. An anti-solvent can be optionally added. The suitable anti-solvent comprises aliphatic ethers, preferably methyl tert-butyl ether.

Another aspect of the invention relates to a process for preparing rasagiline maleate crystalline Form I, said process comprising dissolving or slurrying rasagiline maleate in a suitable solvent, and removing the solvent when necessary.

The suitable solvents comprise ketones, C₁-C₅ alcohols, aliphatic ethers, C₁-C₅ esters, halogenated aliphatic hydrocarbons, water and mixtures thereof. The preferred ketones are acetone, 2-butanone and methyl isobutyl ketone. The preferred C₁-C₅ alcohols are methanol and ethanol. The preferred aliphatic ethers are methyl tert-butyl ether and tetrahydrofuran. The preferred C₁-C₅ ester is isopropyl acetate. The preferred halogenated aliphatic hydrocarbon is chloroform.

Another aspect of the present invention includes rasagiline succinate salt and processes for its preparation and isolation.

Another aspect of the present invention relates to a new rasagiline succinate crystalline Form I and processes for obtaining it.

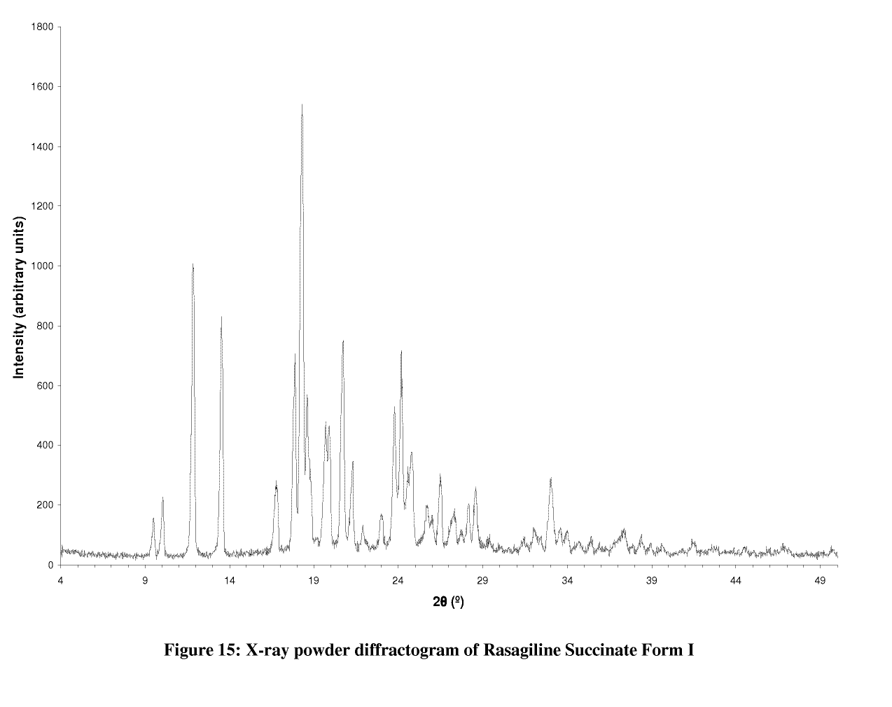

The rasagiline succinate crystalline Form I of the present invention shows an XRD pattern (2θ) (±0.2°) having characteristic peaks at approximately: 10.1, 11.8, 13.5, 16.8, 17.9, 18.3, 18.6, 19.7, 19.9, 20.7, 21.3, 23.8, 24.2, 24.8, 26.5, 28.6, 33.0° with further peaks at: 9.5, 23.0, 25.7, 27.3, 28.2°. FIG. 15 illustrates the XRD of rasagiline succinate crystalline Form I.

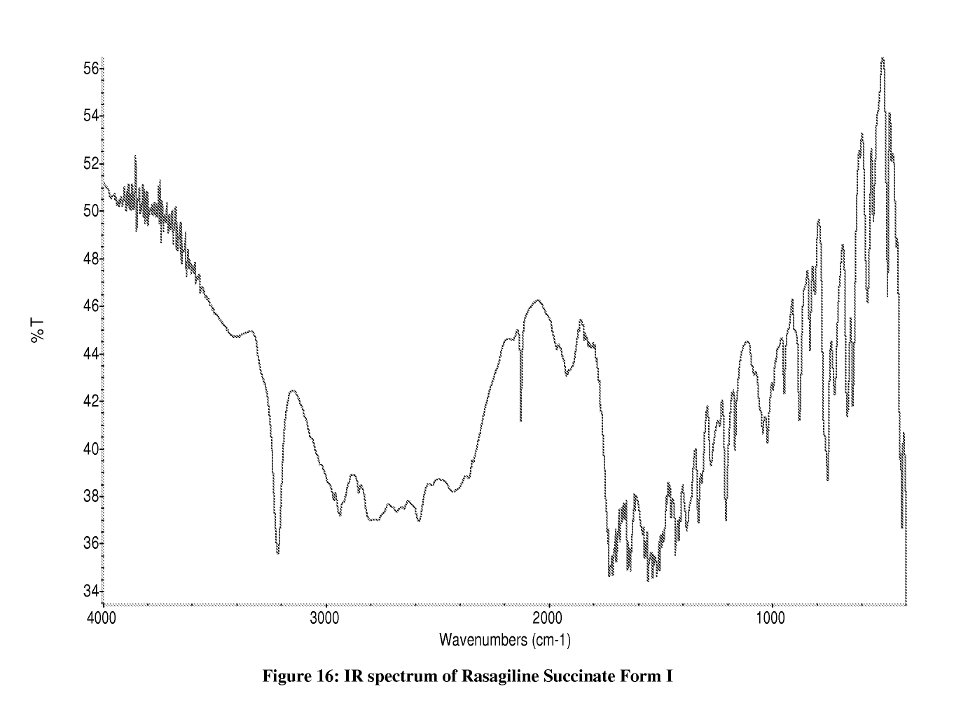

The rasagiline succinate crystalline Form I of the present invention shows an IR spectrum having characteristic peaks at approximately: 3216.9, 2584.9, 2126.8, 1922.9, 1731.4, 1633.8, 1556.8, 1434.5, 1384.6, 1331.5, 1275.1, 1207.5, 1167.3, 1020.6, 945.6, 876.5, 831.7, 752.2, 719.5, 661.8, 639.6, 572.9, 547.6, 483.9, 418.9 cm⁻¹. FIG. 16 illustrates the IR spectrum of rasagiline succinate crystalline Form I.

The 1:1 salt correlation of rasagiline succinate was confirmed by ¹H NMR spectrum.

The rasagiline succinate Form I of the invention has an excellent flowability, as indicated by a Hausner ratio of about 1.04. In addition, the rasagiline succinate Form I of the invention has a purity higher than about 99.3% relative peak area by HPLC. Also, the rasagiline succinate Form I of the present invention has been found to be highly stable in terms of visual aspect, chemical purity and of polymorphic form after ten months of storage and after being submitted to accelerated stability conditions (40° C. and 75% RH) for one and two months. Further, the rasagiline succinate Form I of the invention is very soluble in water, (i.e. solubility>1200 g/L).

Another aspect of the invention relates to a process for preparing rasagiline succinate salt Form I, said process comprising contacting rasagiline base with succinic acid, optionally in the presence of a suitable solvent, and removing the solvent when necessary.

The suitable solvents comprise C₁-C₅ alcohols, water and mixtures thereof. The preferred C₁-C₅ alcohol is 2-propanol. An anti-solvent can be optionally added. The suitable anti-solvent comprises aliphatic ethers, preferably methyl tert-butyl ether.

Preferably, the process for preparing rasagiline succinate Form I comprises contacting rasagiline base with succinic acid in the presence of a suitable solvent, and removing the solvent. More preferably, the suitable solvent is a C₁-C₅ alcohol solvent, and even more preferably is 2-propanol.

Another aspect of the invention relates to a process for preparing rasagiline succinate crystalline Form I, said process comprising dissolving or slurrying rasagiline succinate in a suitable solvent, and removing the solvent when necessary.

The suitable solvents comprise ketones, C₁-C₅ alcohols, aliphatic ethers, C₁-C₅ esters, halogenated aliphatic hydrocarbons, water and mixtures thereof. The preferred ketones are acetone, 2-butanone and methyl isobutylketone. The preferred C₁-C₅ alcohols are methanol and ethanol. The preferred aliphatic ethers are methyl tert-butyl ether and tetrahydrofuran. The preferred C₁-C₅ ester is isopropyl acetate. The preferred halogenated aliphatic hydrocarbon is chloroform.

Another aspect of the present invention relates to a new rasagiline acetate crystalline Form I and processes for obtaining it.

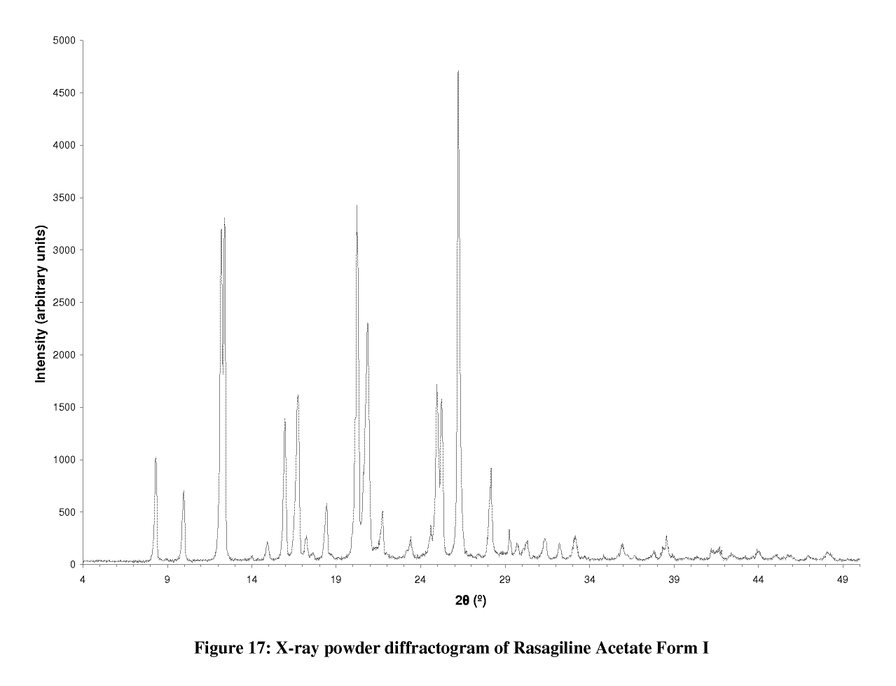

The rasagiline acetate crystalline Form I of the present invention shows an XRD pattern (2θ) (±0.2°) having characteristic peaks at approximately: 8.3, 10.0, 12.2, 12.4, 16.0, 16.7, 18.4, 20.2, 20.8, 25.0, 25.2, 26.2, 28.2° with further peaks at: 21.7, 29.2°. FIG. 17 illustrates the XRD of rasagiline acetate crystalline Form I.

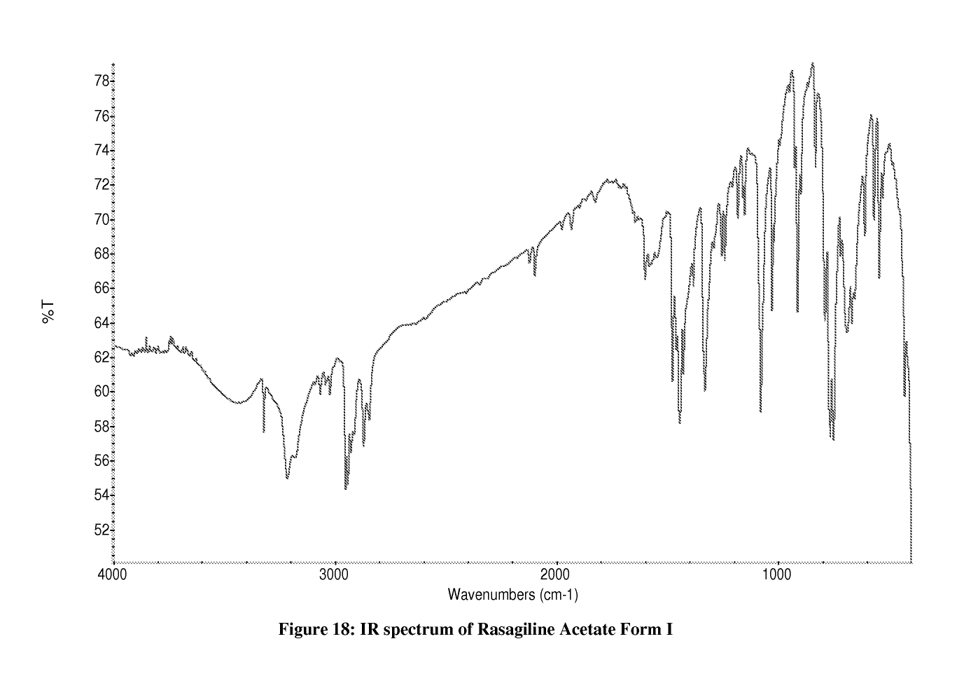

The rasagiline acetate Form I crystalline of the present invention shows an IR spectrum having characteristic peaks at approximately: 3428.0, 3322.9, 3216.1, 3068.3, 3023.2, 2952.2, 2942.8, 2929.3, 2871.8, 1478.1, 1446.8, 1428.5, 1331.8, 1079.0, 765.8, 751.6 cm⁻¹ with further peaks at: 2098.9, 1602.3, 1384.4, 1256.5, 1242.2, 1182.7, 1151.6, 1029.0, 913.9, 788.9, 690.6, 668.1, 610.8, 567.6, 545.8, 429.8 cm⁻¹. FIG. 18 illustrates the IR spectrum of rasagiline acetate crystalline Form I.

The rasagiline acetate Form I of the invention has a purity higher than about 98.0% relative peak area by HPLC.

Another aspect of the invention relates to a process for preparing rasagiline acetate crystalline Form I, said process comprising dissolving or slurrying rasagiline acetate in a suitable solvent, and removing the solvent when necessary.

The suitable solvents comprise ketones, C₁-C₅ alcohols, aliphatic ethers, C₁-C₅ esters, halogenated aliphatic hydrocarbons, water and mixtures thereof. The preferred ketones are acetone, 2-butanone and methyl isobutylketone. The preferred C₁-C₅ alcohols are methanol and ethanol. The preferred aliphatic ethers are methyl tert-butyl ether and tetrahydrofuran. The preferred C₁-C₅ ester is isopropyl acetate. The preferred halogenated aliphatic hydrocarbon is chloroform.

Another aspect of the present invention relates to a new rasagiline acetate crystalline Form II and processes for obtaining it.

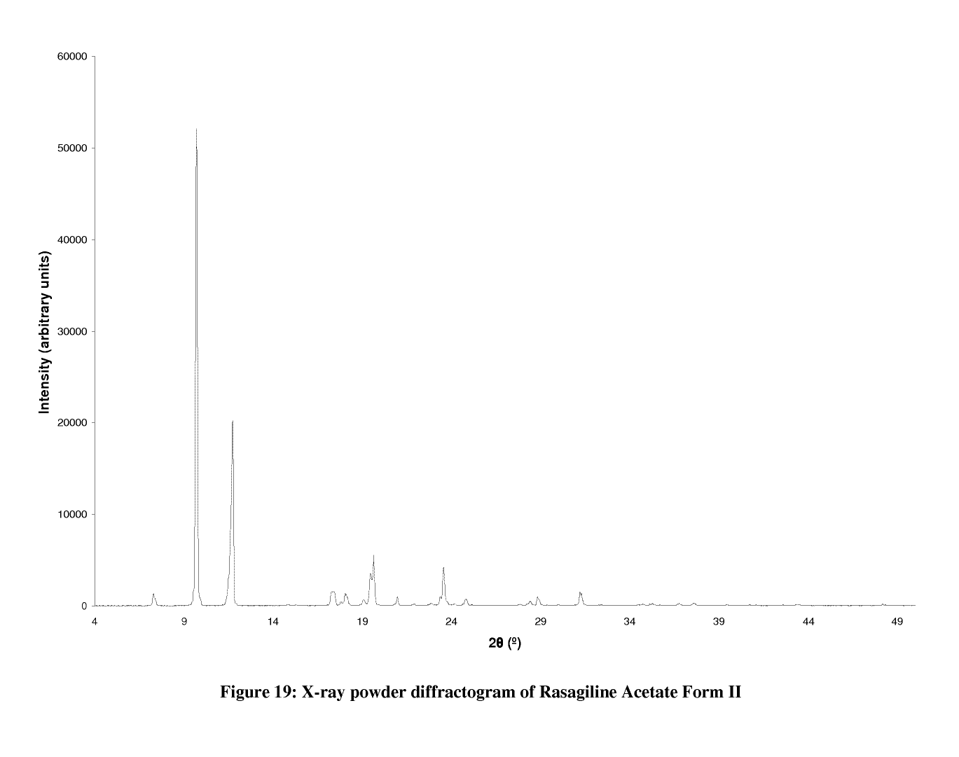

The rasagiline acetate crystalline Form II of the present invention shows an XRD pattern (2θ) (±0.2°) having characteristic peaks at approximately: 7.3, 9.7, 11.7, 17.4, 18.0, 19.5, 19.6, 23.6, 31.2° and with further peaks at: 17.8, 19.1, 21.0, 23.4, 24.8, 28.4, 28.8°. FIG. 19 illustrates the XRD of rasagiline acetate crystalline Form II.

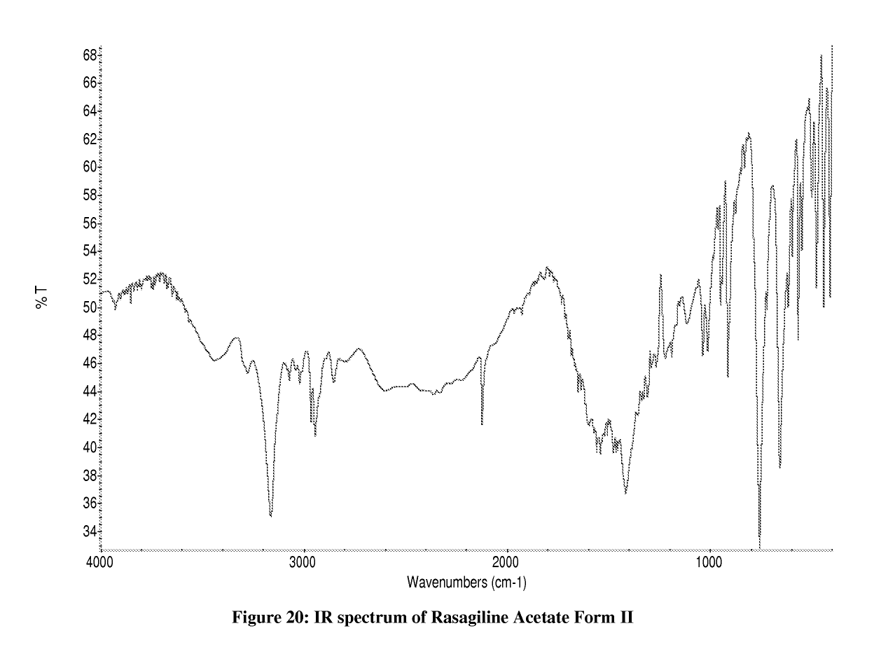

The rasagiline acetate Form II crystalline of the present invention shows an IR spectrum having characteristic peaks at approximately: 3445.8, 3164.0, 3021.6, 2966.1, 2945.6, 2852.8, 2359.0, 2122.4, 1540.1, 1417.9, 1220.6, 1036.9, 1012.3, 914.0, 758.0, 657.3, 567.2 cm⁻¹ and with further peaks at: 1112.7, 947.4, 616.3, 596.2, 549.7, 477.8, 441.6, 411.2 cm⁻¹. FIG. 20 illustrates the IR spectrum of rasagiline acetate crystalline Form II.

The rasagiline acetate Form II of the invention has a purity higher than about 97.0% relative peak area by HPLC.

Another aspect of the invention relates to a process for preparing rasagiline acetate crystalline Form II, said process comprising contacting rasagiline base with acetic acid, optionally in the presence of a suitable solvent, and removing the solvent when necessary.

The suitable solvents comprise at least a C₁-C₅ alcohol solvent, water and mixtures thereof. The preferred C₁-C₅alcohol solvent is 2-propanol. An anti-solvent can be optionally added. The suitable anti-solvent comprises aliphatic ethers, preferably methyl tert-butyl ether.

Another aspect of the present invention relates to a new rasagiline L-tartrate crystalline Form I and processes for obtaining it.

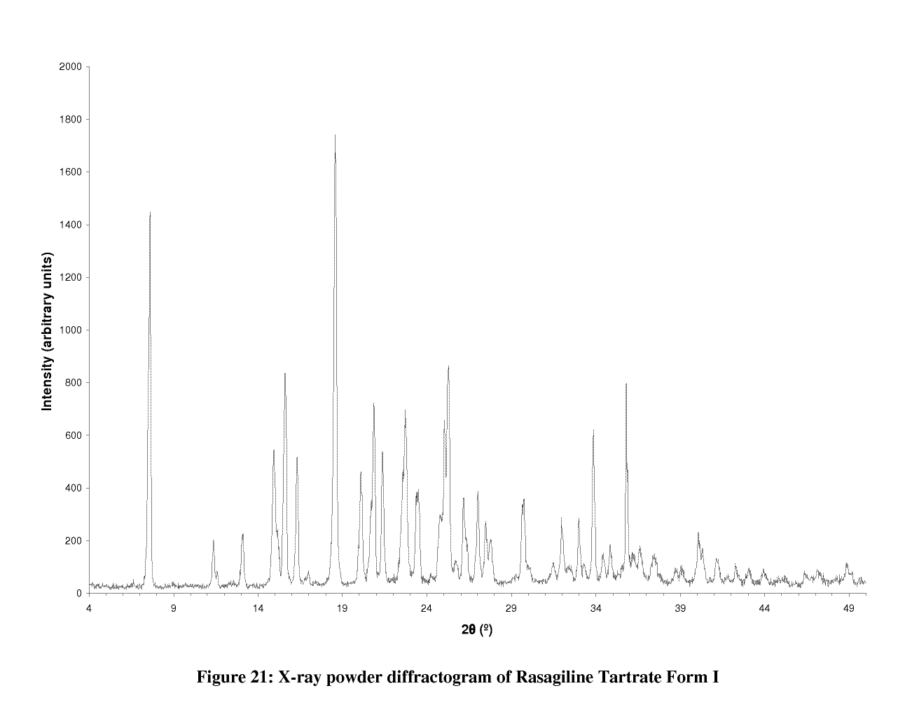

The rasagiline L-tartrate crystalline Form I of the present invention shows an XRD pattern (2θ) (±0.2°) having characteristic peaks at approximately: 7.6, 14.9, 15.6, 16.3, 18.6, 20.1, 20.8, 21.4, 22.6, 22.7, 25.1, 25.3, 33.9, 35.8° with further peaks at: 11.4, 13.1, 23.4, 24.8, 26.2, 27.0, 27.5, 27.8, 29.7, 32.0, 33.0, 34.4, 34.8, 40.1°. FIG. 21 illustrates the XRD of rasagiline L-tartrate crystalline Form I.

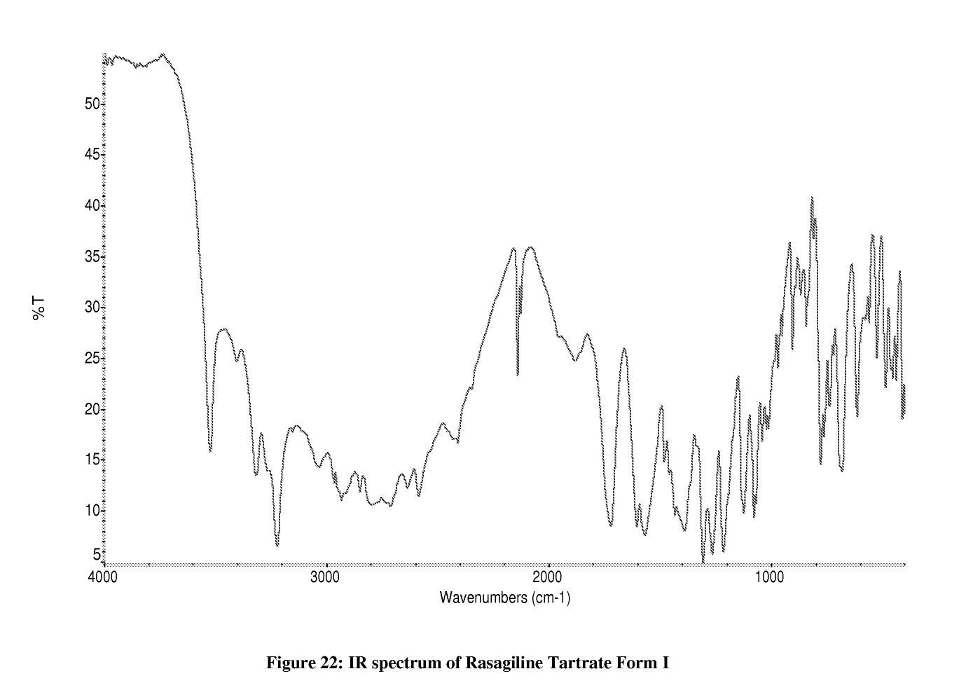

The rasagiline L-tartrate crystalline Form I of the present invention shows an IR spectrum having characteristic peaks at approximately: 3524.3, 3318.3, 3220.8, 2933.4, 2711.7, 2585.5, 2410.3, 1722.0, 1604.2, 1568.1, 1481.0, 1390.6, 1307.6, 1264.8, 1215.4, 1123.6, 1077.4, 1041.6, 1023.3, 777.6, 763.6, 682.5 cm⁻¹ with further peaks at: 2141.7, 2125.9, 1881.0, 971.0, 951.5, 905.1, 867.5, 842.6, 736.3, 614.1, 559.8, 524.0, 485.3, 453.7, 437.1, 410.8 cm⁻¹. FIG. 22 illustrates the IR spectrum of rasagiline L-tartrate crystalline Form I.

The 1:1 salt correlation of rasagiline L-tartrate was confirmed by ¹H NMR spectrum.

The rasagiline L-tartrate Form I of the invention has a purity higher than about 99.5% relative peak area by HPLC. Also, the rasagiline L-tartrate Form I of the present invention has been found to be highly stable in terms of polymorphic form after ten months of storage.

Another aspect of the invention relates to a process for preparing rasagiline L-tartrate Form I, said process comprising dissolving or slurrying rasagiline tartrate in a suitable solvent, and removing the solvent when necessary.

The suitable solvents comprise ketones, C₁-C₅ alcohols, aliphatic ethers, C₁-C₅ esters, halogenated aliphatic hydrocarbons, water and mixtures thereof. The preferred ketones are acetone, 2-butanone and methyl isobutylketone. The preferred C₁-C₅ alcohols are methanol and ethanol. The preferred aliphatic ethers are methyl tert-butyl ether and tetrahydrofuran. The preferred C₁-C₅ ester is isopropyl acetate. The preferred halogenated aliphatic hydrocarbon is chloroform.

Another aspect of the present invention relates to a new rasagiline L-hemitartrate crystalline Form I and processes for obtaining it.

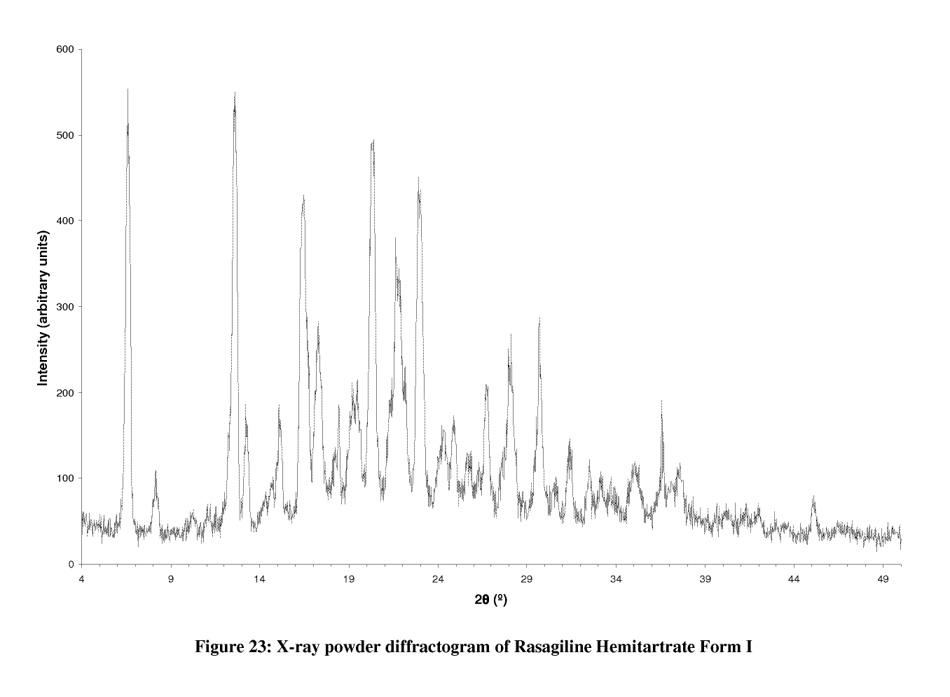

The rasagiline L-hemitartrate crystalline Form I of the present invention shows an XRD pattern (2θ) (±0.2°) having characteristic peaks at approximately: 6.6, 12.6, 16.5, 20.3, 22.9, 23.0° with further peaks at: 8.1, 13.2, 15.1, 17.3, 18.4, 19.5, 21.6, 21.7, 21.8, 22.2, 22.8, 24.3, 24.9, 26.7, 28.0, 29.6, 31.4, 32.5, 36.5°. FIG. 23 illustrates the XRD of rasagiline L-hemitartrate crystalline Form I.

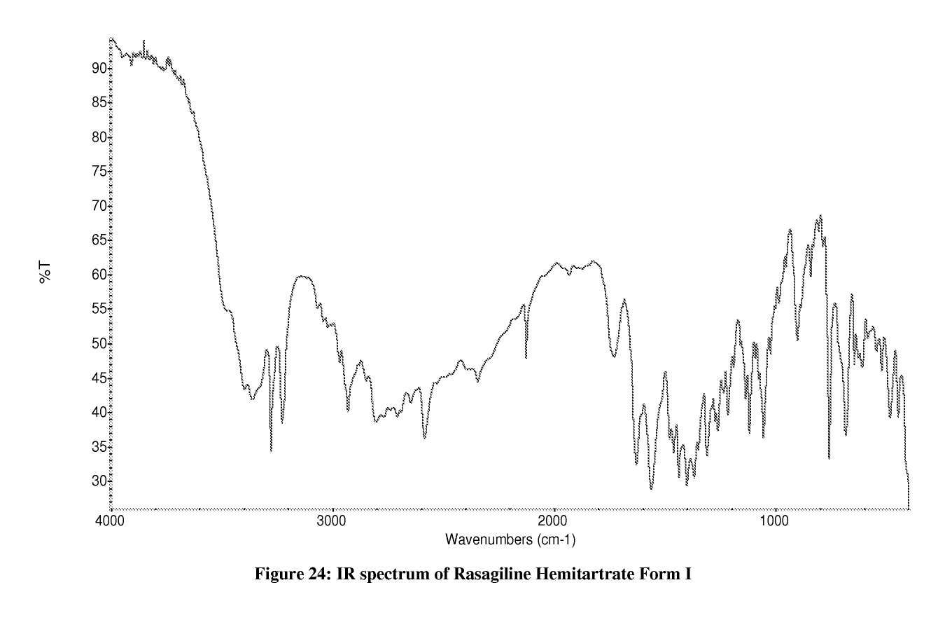

The rasagiline L-hemitartrate crystalline Form I of the present invention shows an IR spectrum having characteristic peaks at approximately: 3362.6, 3279.6, 3228.5, 2931.6, 2805.0, 2585.5, 1629.8, 1562.5, 1462.2, 1439.1, 1401.4, 1368.1, 1312.0, 1261.9, 1216.6, 1135.3, 1118.9, 1056.1, 758.8, 686.0, 484.7, 447.2 cm⁻¹ with further peaks at: 2346.8, 2126.9, 1729.9, 903.6, 842.9, 645.9, 611.5, 522.0 cm⁻¹. FIG. 24 illustrates the IR spectrum of rasagiline L-hemitartrate crystalline Form I.

The 2:1 salt correlation of rasagiline L-hemitartrate was confirmed by ¹H NMR spectrum.

The rasagiline L-hemitartrate Form I of the invention has a passable flowability, as indicated by a Hausner ratio equal to 1.31. In addition, the rasagiline L-hemitartrate Form I of the invention has a purity higher than about 99.1% relative peak area by HPLC. Also, the rasagiline L-hemitartrate Form I of the present invention has been found to be highly stable in terms of visual aspect, chemical purity and of polymorphic form after ten months of storage and after being submitted to accelerated stability conditions (40° C. and 75% RH) for one and two months. Further, the rasagiline L-hemitartrate Form I of the invention is soluble in water (i.e. solubility=about 59 g/L).

Another aspect of the invention relates to a process for preparing rasagiline L-hemitartrate crystalline Form I, said process comprising contacting rasagiline base with a suitable amount of L-tartaric acid, optionally in the presence of a suitable solvent, and removing the solvent when necessary.

Preferably, the process for preparing rasagiline hemitartrate Form I comprises contacting rasagiline base with a suitable amount of L-tartaric acid in the presence of a suitable solvent, and removing the solvent.

The suitable solvents comprise at least a C₁-C₅ alcohol, water and mixtures thereof. The preferred C₁-C₅ alcohol is 2-propanol. An anti-solvent can be optionally added. The suitable anti-solvent comprises aliphatic ethers, preferably methyl tert-butyl ether.

Another aspect of the present invention relates to a new rasagiline fumarate crystalline Form I and processes for obtaining it.

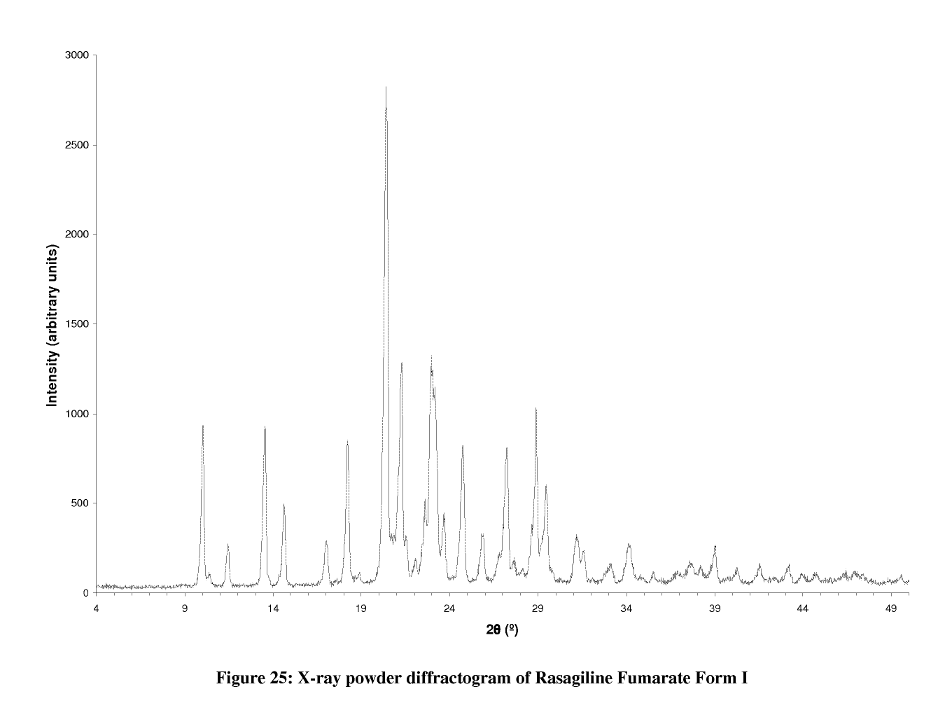

The rasagiline fumarate crystalline Form I of the present invention shows an XRD pattern (2θ) (±0.2°) having characteristic peaks at approximately: 10.0, 13.5, 14.6, 18.2, 20.4, 21.2, 22.6, 23.0, 23.7, 24.7, 27.2, 28.9, 29.5° with further peaks at: 11.5, 17.0, 25.9, 31.2, 31.6, 34.1, 39.0°. FIG. 25 illustrates the XRD of rasagiline fumarate crystalline Form I.

The rasagiline fumarate crystalline Form I of the present invention shows an IR spectrum having characteristic peaks at approximately: 3267.2, 2805.2, 2586.2, 2361.8, 1699.2, 1683.7, 1652.7, 1635.2, 1558.4, 1540.4, 1478.8, 1456.2, 1436.4, 1373.7, 1336.4, 1316.6, 1291.3, 1246.6, 1203.2, 993.7, 763.4, 733.0, 696.3, 640.3, 443.5, 418.4 cm⁻¹ with further peaks at: 3219.4, 2126.6, 1843.5, 1506.6, 1092.3, 1021.4, 963.3, 899.7, 790.2, 564.2, 549.2, 496.1 cm⁻¹.

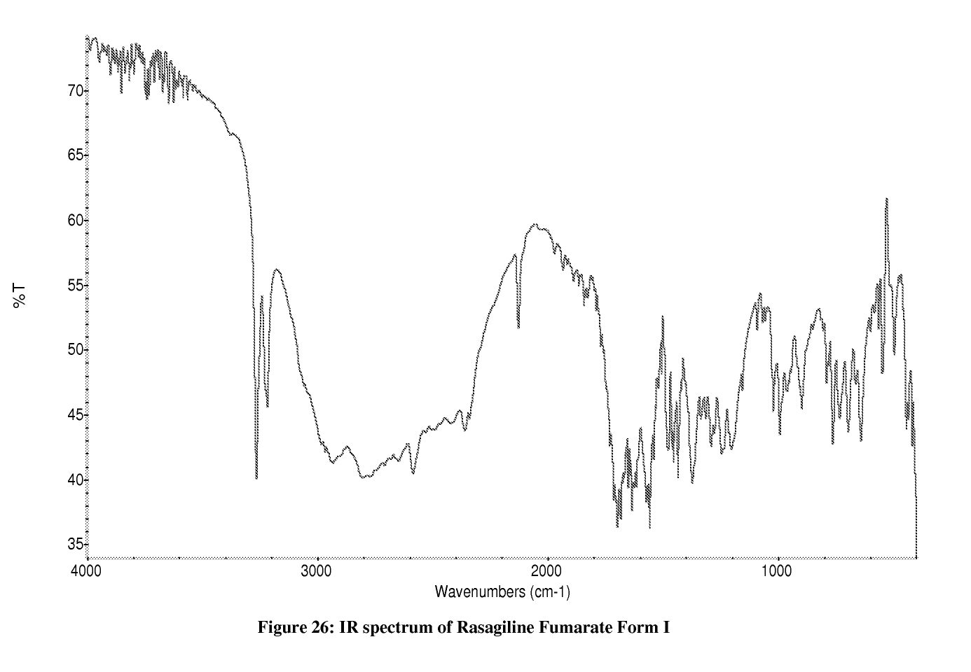

FIG. 26 illustrates the IR spectrum of rasagiline fumarate crystalline Form I.

The 1:1 salt correlation of rasagiline fumarate was confirmed by ¹H NMR spectrum.

Another aspect of the invention relates to a process for preparing rasagiline fumarate salt form, said process comprising contacting rasagiline base with fumaric acid, optionally in the presence of a suitable solvent, and removing the solvent when necessary.

The suitable solvents comprise at least a C₁-C₅ alcohol, water and mixtures thereof. The preferred C₁-C₅ alcohol is 2-propanol. An anti-solvent can be optionally added. The suitable anti-solvent comprises aliphatic ethers, preferably methyl tert-butyl ether.

Another aspect of the invention relates to a process for preparing rasagiline fumarate Form I, said process comprising dissolving or slurrying rasagiline fumarate in a suitable solvent, and removing the solvent when necessary.

The suitable solvents comprise ketones, C₁-C₅ alcohols, aliphatic ethers, C₁-C₅ esters, halogenated aliphatic hydrocarbons, water and mixtures thereof. The preferred ketones are acetone, 2-butanone and methyl isobutylketone. The preferred C₁-C₅ alcohols are methanol and ethanol. The preferred aliphatic ethers are methyl tert-butyl ether and tetrahydrofuran. The preferred C₁-C₅ ester is isopropyl acetate. The preferred halogenated aliphatic hydrocarbon is chloroform.

Another aspect of the present invention relates to a new rasagiline hydrochloride crystalline Form I and processes for obtaining it.

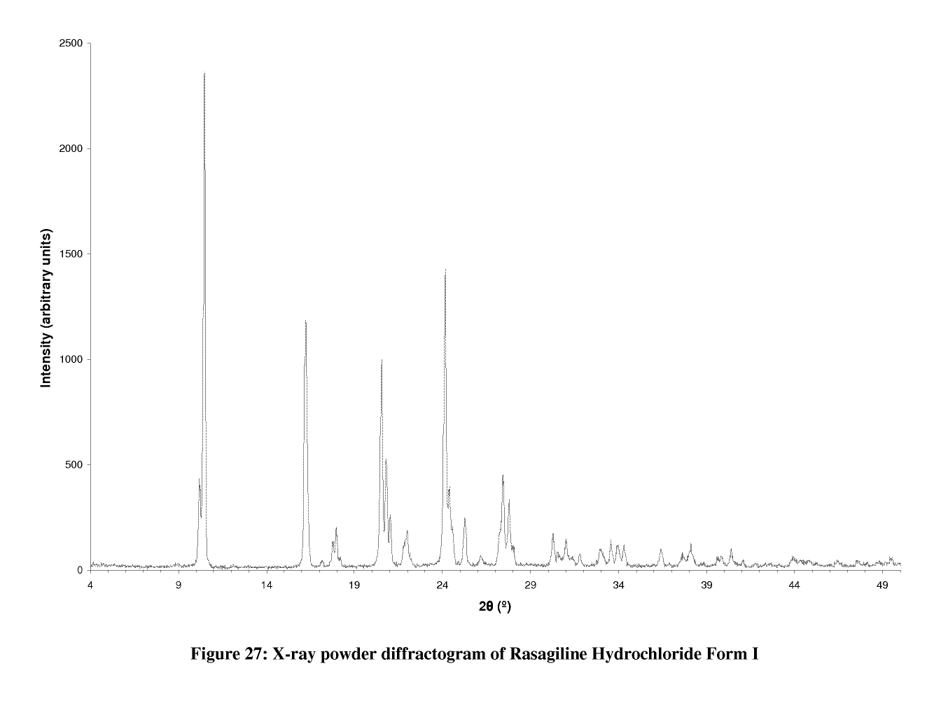

The rasagiline hydrochloride crystalline Form I of the present invention shows an XRD pattern (2θ) (±0.2°) having characteristic peaks at approximately: 10.2, 10.5, 16.2, 18.0, 20.5, 20.8, 21.0, 22.0, 24.1, 24.4, 25.3, 27.4, 27.8, 30.3° with further peaks at: 17.7, 31.0, 33.0, 33.5, 34.0, 34.3, 36.4, 38.1°. FIG. 27 illustrates the XRD of rasagiline hydrochloride crystalline Form I.

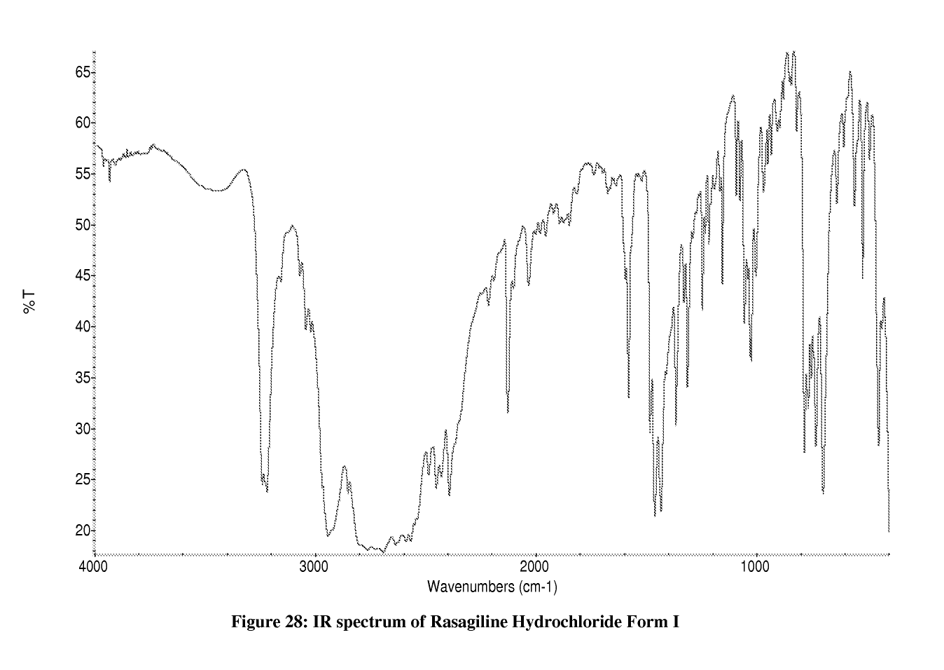

The rasagiline hydrochloride crystalline Form I of the present invention shows an IR spectrum having characteristic peaks at approximately: 3219.2, 3043.8, 2943.3, 2694.0, 2487.5, 2452.4, 2392.8, 2216.5, 2126.5, 2034.0, 1581.9, 1483.2, 1463.0, 1434.1, 1366.9, 1329.6, 1314.2, 1246.5, 1216.6, 1154.8, 1055.9, 1030.9, 1023.3, 1003.0, 783.1, 765.6, 753.6, 730.2, 698.4, 518.5, 447.0 cm⁻¹ with further peaks at: 3445.7, 1848.2, 1674.7, 1092.8, 1075.0, 969.6, 950.0, 933.6, 906.5, 819.3, 636.1, 604.2, 556.4, 488.4 cm⁻¹. FIG. 28 illustrates the IR spectrum of rasagiline hydrochloride crystalline Form I.

The rasagiline hydrochloride Form I of the invention has a purity higher than about 99.1% relative peak area by HPLC. Also, the rasagiline hydrochloride Form I of the present invention has been found to be highly stable in terms of chemical purity and of polymorphic form after ten months of storage.

Another aspect of the invention relates to a process for preparing rasagiline hydrochloride salt Form I, said process comprising contacting rasagiline base with hydrochloric acid in the presence of a solvent comprising a mixture of at least one C₁-C₅ alcohol and water, wherein the C₁-C₅ alcohol/water ratio (v/v) is equal to or higher than 4, and removing the solvent.

The at least one C₁-C₅ alcohol solvent is preferably 2-propanol, and the 2-propanol/water ratio (v/v) is preferably equal to 4.

Another aspect of the present invention relates to a new rasagiline hydrochloride crystalline Form II and processes for obtaining it.

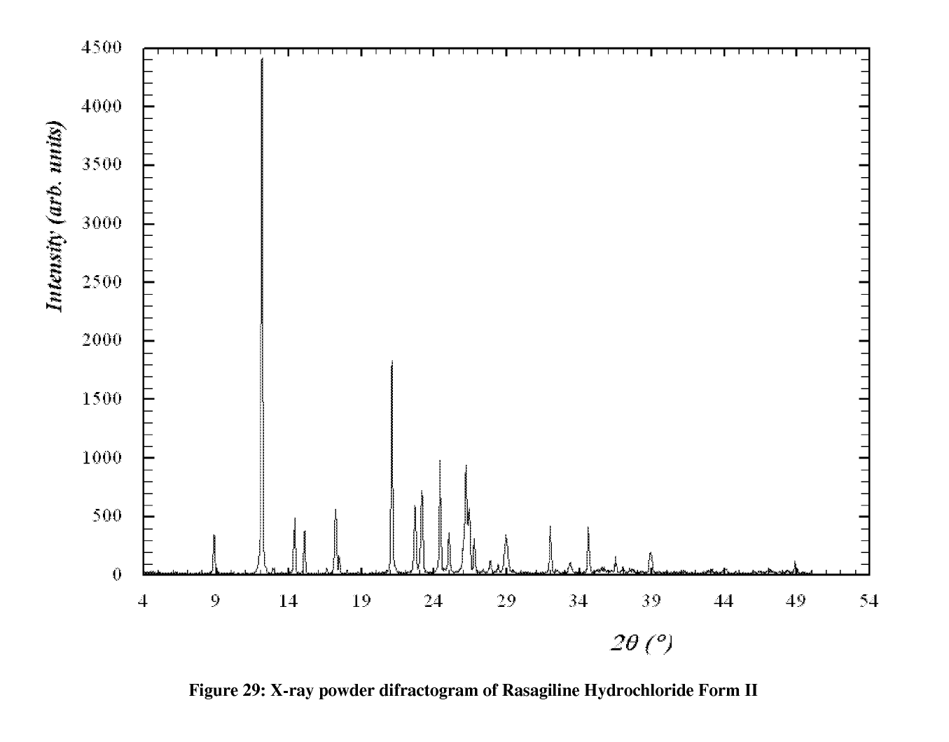

The rasagiline hydrochloride crystalline Form II of the present invention shows an XRD pattern (2θ) (±0.2°) having characteristic peaks at approximately: 8.9, 12.1, 14.4, 15.1, 17.2, 17.5, 21.1, 22.7, 23.1, 24.4, 25.1, 26.2, 26.4, 26.8, 27.9, 29.0, 32.0, 34.6, 36.5, 38.9°. FIG. 29 illustrates the XRD of rasagiline hydrochloride crystalline Form II.

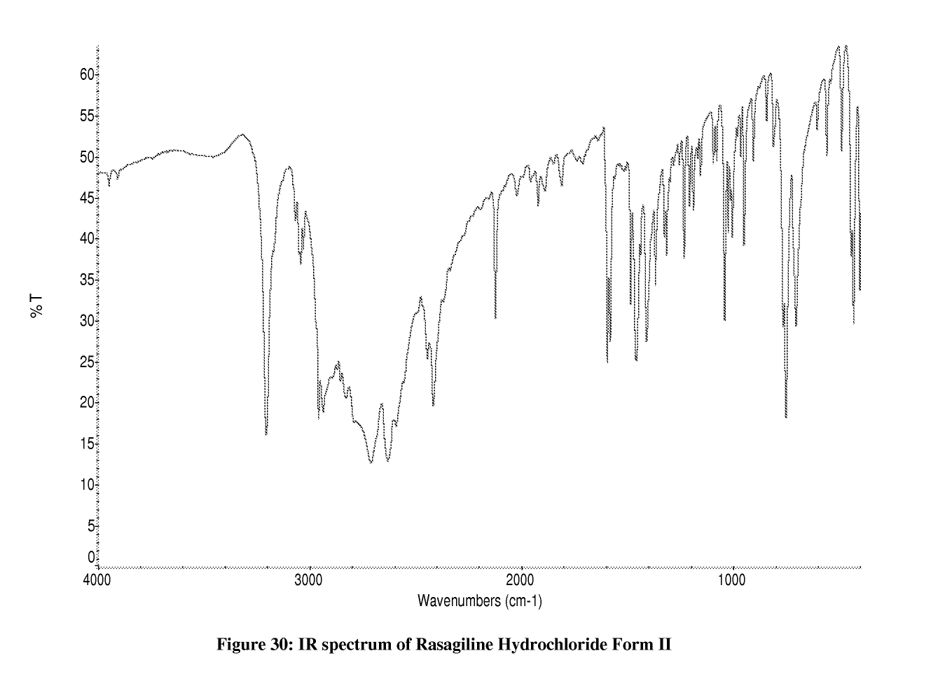

The rasagiline hydrochloride crystalline Form II of the present invention shows an IR spectrum having characteristic peaks at approximately: 3206, 3070, 3044, 3031, 2958, 2937, 2712, 2632, 2445, 2418, 1595, 1583, 1485, 1458, 1409, 1368, 1325, 1316, 1234, 1207, 1189, 1154, 1095, 1078, 1042, 1024, 1005, 949, 966, 906, 843, 811, 765, 752, 704, 603, 559, 489, 443, 432, 401 cm⁻¹. FIG. 30 illustrates the IR spectrum of rasagiline hydrochloride crystalline Form II.

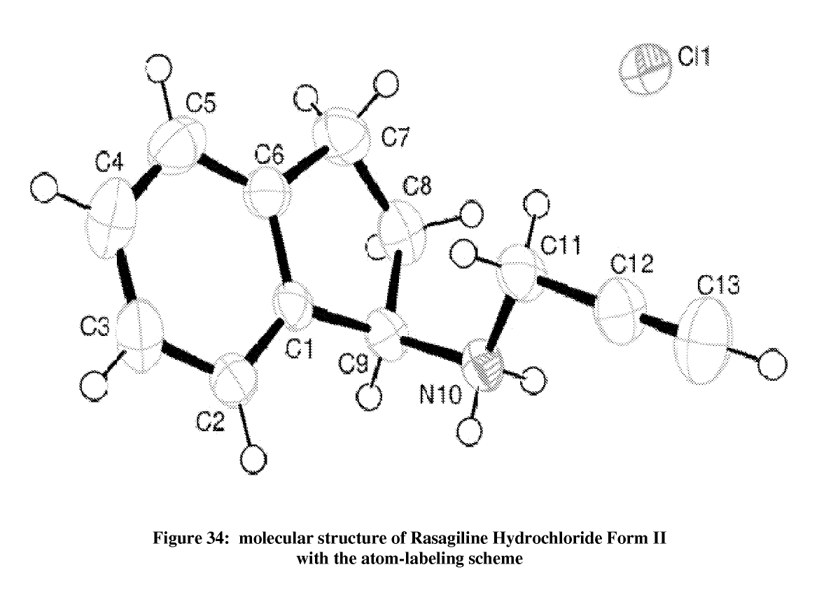

FIG. 34 illustrates the molecular structure of rasagiline hydrochloride Form II with the atom-labeling scheme. The basic crystallographic data for single crystal of rasagiline hydrochloride Form II is as follows:

| | |
|---|---|
| Empirical formula | C₁₂H₁₄Cl₁N₁ |
| Formula weight | 207.69 |
| Temperature | 293(2) K |
| Crystal size | 0.36 × 0.24 × 0.21 mm |
| Crystal system, space group | Orthorhombic, P 2₁2₁2₁ |
| Unit cell dimensions | a = 7.273(2) Å |
| | b = 7.828(2) Å |
| | c = 19.945(6) Å |
| | α = γ = β = 90° |
| Volume | 1135.4(5) Å³ |
| Z | 4 |
| Calculated density | 1.215 mg/cm³ |

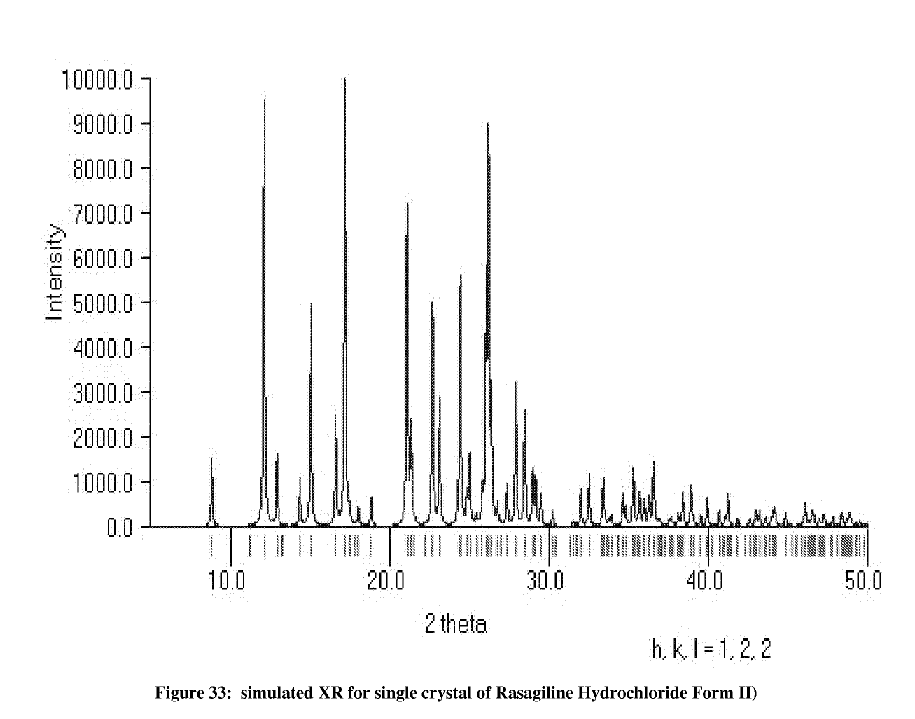

FIG. 33 illustrates a simulated X-ray diffractogram, which has been calculated using the crystallographic data for single crystal of rasagiline hydrochloride Form II. The simulated X-ray diffractogram of FIG. 33 is substantially similar to the X-ray powder diffractogram of rasagiline hydrochloride Form II of FIG. 29.

The rasagiline hydrochloride Form II of the invention has an excellent flowability, as indicated by a Hausner ratio of about 1.00. In addition, the rasagiline hydrochloride Form II of the invention has a purity higher than about 99.9% relative peak area by HPLC. Also, the rasagiline hydrochloride Form II of the present invention has been found to be highly stable in terms of visual aspect, chemical purity and of polymorphic form after being submitted to accelerated stability conditions (40° C. and 75% RH) for one and two months. Further, the rasagiline hydrochloride Form II of the invention is freely soluble in water (i.e. solubility=about 400 g/L).

Another aspect of the invention relates to a process for preparing rasagiline hydrochloride salt Form II, said process comprising contacting rasagiline base with hydrochloric acid in the presence of a solvent comprising a mixture of at least one C₁-C₅ alcohol and water, wherein the C₁-C₅ alcohol/water ratio (v/v) is equal to or less than 3, and removing the solvent.

The at least one C₁-C₅ alcohol solvent is preferably 2-propanol, and the 2-propanol/water ratio (v/v) is preferably equal to 3.

Another aspect of the present invention relates to a new rasagiline besylate crystalline Form I and processes for obtaining it.

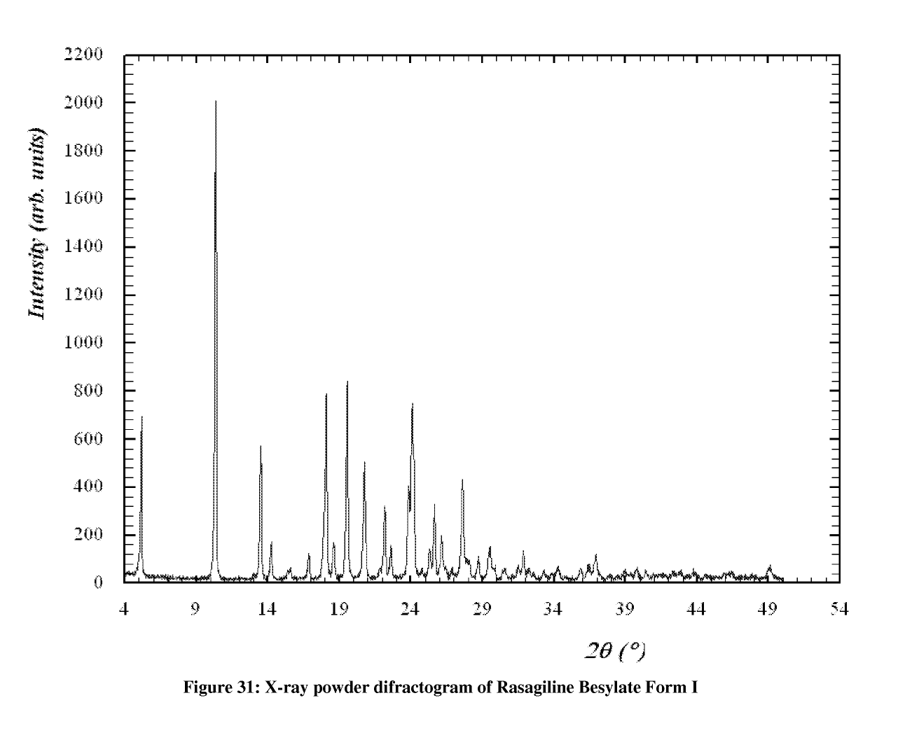

The rasagiline besylate crystalline Form I of the present invention shows an XRD pattern (2θ) (±0.2°) having characteristic peaks at approximately: 5.2, 10.4, 13.5, 14.2, 16.9, 18.1, 18.6, 19.5, 20.7, 22.2, 22.6, 23.8, 24.1, 25.3, 25.6, 26.2, 27.6, 28.7, 29.5°. FIG. 31 illustrates the XRD of rasagiline besylate crystalline Form I.

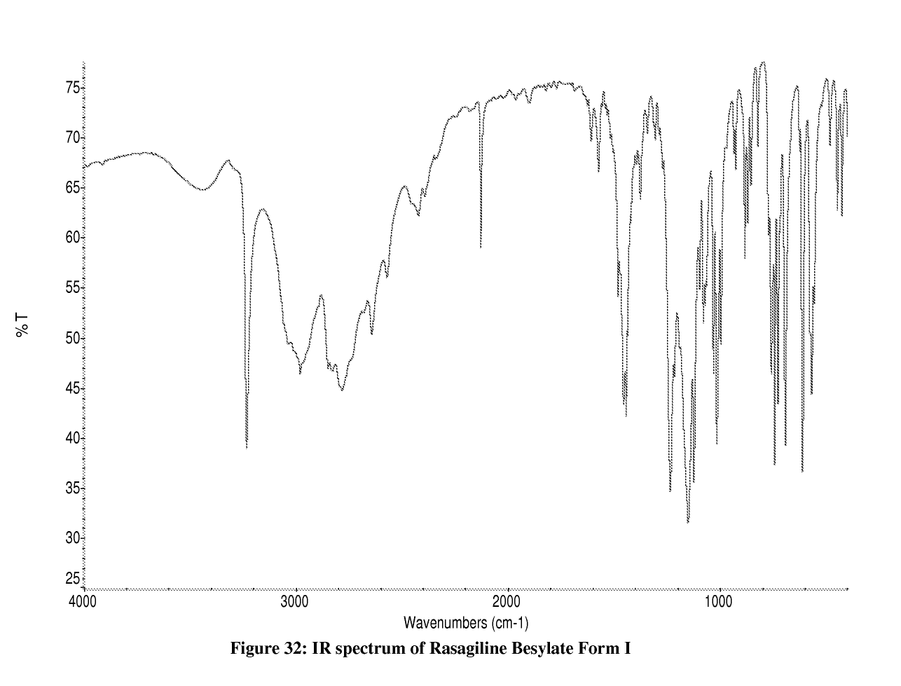

The rasagiline besylate crystalline Form I of the present invention shows an IR spectrum having characteristic peaks at approximately: 3433, 3232, 2981, 2783, 2643, 2572, 2424, 1481, 1456, 1444, 1377, 1236, 1150, 1123, 1098, 1078, 1071, 1032, 1015, 996, 884, 869, 772, 758, 743, 727, 693, 612, 568, 559, 447, 426 cm⁻¹. FIG. 32 illustrates the IR spectrum of rasagiline besylate crystalline Form I.

The rasagiline besylate Form I of the invention has an excellent flowability, as indicated by a Hausner ratio equal to 1.00. In addition, the rasagiline besylate Form I of the invention has a purity higher than about 99.9% relative peak area by HPLC. Also, the rasagiline besylate Form I of the present invention has been found to be stable in terms of chemical purity and of polymorphic form after being submitted to accelerated stability conditions (40° C. and 75% RH) for one month. Further, the rasagiline besylate Form I of the invention is freely soluble in water (i.e. solubility>800 g/L).

Another aspect of the invention relates to a process for preparing rasagiline besylate crystalline Form I, said process comprising contacting rasagiline base with benzenesulfonic acid in the presence of a suitable solvent, and removing the solvent.

Suitable solvents include a C₁-C₅ alcohol, an aromatic hydrocarbon solvent, and mixtures thereof. The preferred C₁-C₅ alcohol is 2-propanol. The preferred aromatic hydrocarbon solvent is toluene.

Another aspect of the invention includes a formulation including the rasagiline salts obtained according to the processes of the invention.

In another further aspect, the invention relates to the use of the acid addition salts of rasagiline of the invention for preparing rasagiline mesylate.

The invention will now be described in more detail by way of examples. The following examples are for illustrative purposes only and are not intended, nor should they be interpreted, to limit the scope of the invention.

### EXAMPLES

### General Experimental Conditions

### HPLC Method

The chromatographic separation was carried out in a Chiralpak IC, 5 μm, 250×4.6 mm I.D column; at 30° C.

The mobile phase was prepared by mixing 950 mL of n-hexane, 40 mL of 2-propanol, 10 mL of ethanol, 4 mL of trifluoroacetic acid and 1 mL of diethylamine. The mixture was mixed thoroughly.

The chromatograph was equipped with a 265 nm detector and the flow rate was 1.4 mL per minute.

The test samples were prepared by dissolving the appropriate amount of sample to obtain 10 mg per mL in diluent. For the majority of the rasagiline salts, the diluent was prepared by mixing 89 mL of mobile phase, 10 mL of 2-propanol and 1 mL of diethylamine. For the rasagiline succinate, the rasagiline hydrochloride, the rasagiline L-hemitartrate, and the rasagiline besylate salts, the diluent was prepared by mixing 49 mL of mobile phase, 50 mL of ethanol and 1 mL diethylamine. The injection volume was 5 μL.

### X-Ray Powder Diffraction (XRD)

The XRD diffractograms were obtained using a RX SIEMENS D5000 diffractometer with a vertical goniometer, a copper anodic tube, and radiation CuKα, λ=1, 54056 Å.

### Single Crystal X-Ray Analysis

X-ray data for a single crystal of rasagiline hydrochloride Form II was collected at 293(2)K on an Enraf-Nonius CAD 4 diffractometer using Mo-K_{□} radiation.

### Infrared Spectra (IR)

Fourier transform IR spectra were acquired on a Thermo Nicolet Nexus spectrometer, and samples were characterized in potassium bromide pellets.

### Particle Size Distribution Method

The particle size for rasagiline salts was measured using a Malvern Mastersizer S particle size analyzer with an MS1-Small Volume Sample Dispersion Unit stirred cell. A 300RF mm lens and a beam length of 2.4 mm were used. Samples of rasagiline salts were suspended in Isopar G containing Soybean Lecithin (1.5 g in 200 mL of Isopar G). The suspensions were mixed and then sonicated for 3 minutes to thoroughly disperse the rasagiline salt particles. Volume distributions were obtained for three times. After completing the measurements, the sample cell was emptied and cleaned, refilled with suspending medium, and the sampling procedure repeated again. For characterization, the values of D₁₀, D₅₀ and D₉₀ (by volume) were specifically listed, each one being the mean of the nine values available for each characterization parameter.

The notation D_{X} [also written as D(v, 0.X)] means that X % of the particles have a diameter less than a specified diameter D. Thus a D₉₀ [or D(v, 0.9)] of 100 μm means that 90% of the particles have a diameter less than 100 μm.

### SPECIFIC EXAMPLES

### Example 1

### Preparation of Rasagiline Benzoate Form I

150 mg of rasagiline base was dissolved in 1 mL of 2-propanol. Benzoic acid (107 mg) was added and the mixture was stirred for 1 h at 40° C. The mixture was allowed to cool to ambient temperature and stirred for 24 hours at this temperature. The mixture was filtered and dried at under ambient conditions.

Analytical data: XRD: Form I, see FIG. 1. IR: see FIG. 2.

### Example 2

### Preparation of Rasagiline Galactarate Form I

150 mg of rasagiline base was dissolved in 1 mL of 2-propanol. Galactaric acid (184 mg) was added and the mixture was stirred for 1 h at 40° C. The mixture was allowed to cool to ambient temperature and stirred for 24 hours at this temperature. The mixture was filtered and dried at under ambient conditions.

Analytical data: XRD: Form I, see FIG. 3. IR: see FIG. 4.

### Example 3

### Preparation of Rasagiline Gluconate Amorphous Form

150 mg of rasagiline base was dissolved in 1 mL of 2-propanol. Gluconic acid (343 mg, 50% aqueous) was added and the mixture was stirred for 1 h at 40° C. The mixture was allowed to cool to ambient temperature and stirred for 24 hours at this temperature. MTBE (1 mL) was added and the mixture stirred for an additional 24 hours. The mixture was concentrated by evaporation under ambient conditions.

Analytical data: XRD: Amorphous form, see FIG. 5. IR: see FIG. 6.

### Example 4

### Preparation of Rasagiline Glucuronate Amorphous Form

150 mg of rasagiline base was dissolved in 1 mL of 2-propanol. D-glucuronic acid (184 mg) was added and the mixture was stirred for 1 h at 40° C. The mixture was allowed to cool to ambient temperature and stirred for 24 hours at this temperature. The mixture was filtered and dried at under ambient conditions.

Analytical data: XRD: Amorphous form, see FIG. 7. IR: see FIG. 8.

### Example 5

### Preparation of Rasagiline Tosylate Form I

150 mg of rasagiline base was dissolved in 1 mL of 2-propanol. p-Toluene sulfonic acid (167 mg) was added, and the mixture was stirred for 1 h at 40° C. The mixture was allowed to cool to ambient temperature and stirred for 24 hours at this temperature. The mixture was concentrated by evaporation under ambient conditions.

Analytical data: XRD: Form I, see FIG. 9. IR: see FIG. 10.

### Example 6

### Preparation of Rasagiline Phosphate Amorphous Form

150 mg of rasagiline base was dissolved in 1 mL of 2-propanol. Phosphoric acid (86 mg 85% aqueous) was added and the mixture was stirred for 1 h at 40° C. The mixture was allowed to cool to ambient temperature and stirred for 48 hours at this temperature. The mixture was concentrated by evaporation under ambient conditions.

Analytical data: XRD: Amorphous form, see FIG. 11. IR: see FIG. 12.

### Example 7

### Preparation of Rasagiline Maleate Form I

Rasagiline base (1.2 g) was dissolved in 2-propanol (7.7 mL). Maleic acid (1.22 g) was added and the mixture was stirred for 2 h at 40° C. The mixture was allowed to cool to ambient temperature and stirred for 24 hours at this temperature. The mixture was filtered and dried under vacuum at 40° C. Yield 2.3 g.

Analytical data: XRD: Form I, see FIG. 13. IR: see FIG. 14.

### Examples 8-17

### Preparation of Rasagiline Maleate Form I

General procedure: Rasagiline maleate (150 mg) was heated in a solvent at reflux, and then allowed to cool to ambient temperature and stirred for 24 hours at this temperature before evaporation of the solvent. This procedure was used for the solvents indicated in Table 2 below.

TABLE 2

| | | | Aspect at | Aspect at | |
|---|---|---|---|---|---|
| Example | Solvent | Quantity | reflux | ambient | Result |
| 8 | Acetone | 0.5 mL | solution | solution | Form I |
| 9 | chloroform | 30.6 mL | solution | solution | Form I |
| 10 | methanol | 0.5 mL | solution | solution | Form I |
| 11 | MTBE | 0.6 mL | solution | suspension | Form I |
| 12 | THF | 0.7 mL | solution | solution | Form I |
| 13 | Ethanol | 0.5 mL | solution | solution | Form I |
| 14 | 2-butanone | 0.5 mL | solution | solution | Form I |
| 15 | Methyl i- | 0.8 mL | solution | suspension | Form I |
| | butylketone | | | | |
| 16 | water/ethanol | 0.4 mL | solution | solution | Form I |
| | (20-80) | | | | |
| 17 | i-propyl acetate | 1.4 mL | solution | suspension | Form I |

### Example 18

### Preparation of Rasagiline Succinate Form I

Rasagiline base (1.78 g) was dissolved in 2-propanol (7.6 mL). Succinic acid (1.23 g) was added and the mixture was stirred for 2 h at 40° C. The mixture was allowed to cool to ambient temperature and stirred for 24 hours at this temperature. The mixture was filtered and dried under vacuum at 40° C. Yield 2.08 g.

Analytical data: XRD: Form I, see FIG. 15. IR: see FIG. 16.

### Examples 19-28

### Preparation of Rasagiline Succinate Form I

General procedure: Rasagiline succinate (150 mg) was heated in a solvent at reflux, and then allowed to cool to ambient temperature and stirred for 24 hours at this temperature before evaporation of the solvent. This procedure was used for the solvents indicated in Table 3 below.

TABLE 3

| | | | Aspect at | Aspect at | |
|---|---|---|---|---|---|
| Examples | Solvent | Quantity | reflux | ambient | Result |
| 19 | acetone | 0.5 mL | solution | solution | Form I |
| 20 | chloroform | 3 mL | suspension | suspension | Form I |
| 21 | methanol | 0.5 mL | solution | solution | Form I |
| 22 | MTBE | 1 mL | solution | suspension | Form I |
| 23 | THF | 0.6 mL | solution | solution | Form I |
| 24 | ethanol | 0.7 mL | solution | solution | Form I |
| 25 | 2-butanone | 0.6 mL | solution | solution | Form I |
| 26 | Methyl i- | 0.8 mL | solution | suspension | Form I |
| | butylketone | | | | |
| 27 | water/ethanol | 0.5 mL | solution | solution | Form I |
| | (20-80) | | | | |
| 28 | i-propyl acetate | 1 mL | solution | suspension | Form I |

### Example 29

### Preparation of Rasagiline Acetate Mixture of Forms I and II

Rasagiline base (2.23 g) was dissolved in 2-propanol (9.5 mL). Acetic acid (0.78 g) was added and the mixture was stirred for 2 h at 40° C. The mixture was allowed to cool to ambient temperature and stirred for 24 hours at this temperature. The mixture was filtered and dried under vacuum at 40° C. Yield 1.82 g.

### Examples 30-39

### Preparation of Rasagiline Acetate Form I

General procedure: Rasagiline acetate (150 mg) was heated in a solvent at reflux, and then allowed to cool to ambient temperature and stirred for 24 hours at this temperature before evaporation of the solvent. This procedure was used for the solvents indicated in Table 4 below.

Analytical data: XRD: Form I, see FIG. 17. IR: see FIG. 18.

TABLE 4

| | | | Aspect at | Aspect at | |
|---|---|---|---|---|---|
| Example | Solvent | Quantity | reflux | ambient | Result |
| 30 | acetone | 0.3 mL | solution | solution | Form I |
| 31 | chloroform | 0.1 mL | solution | solution | Form I |
| 32 | methanol | 0.2 mL | solution | solution | Form I |
| 33 | MTBE | 0.3 mL | solution | suspension | Form I |
| 34 | THF | 0.2 mL | solution | solution | Form I |
| 35 | ethanol | 0.2 mL | solution | solution | Form I |
| 36 | 2-butanone | 0.2 mL | solution | suspension | Form I |
| 37 | Methyl i- | 0.2 mL | solution | solution | Form I |
| | butylketone | | | | |
| 38 | water/ethanol | 0.2 mL | solution | solution | Form I |
| | (20-80) | | | | |
| 39 | i-propyl acetate | 0.2 mL | solution | suspension | Form I |

### Example 40

### Preparation of Rasagiline Acetate Form II

Rasagiline base (150 mg) was dissolved in 2-propanol (1 mL). Acetic acid (53 mg) was added and the mixture was stirred for 1 h at 40° C. The mixture was allowed to cool to ambient temperature and stirred for 24 hours at this temperature. The mixture was filtered and dried under ambient conditions.

Analytical data: XRD: Form II, see FIG. 19. IR: see FIG. 20.

### Examples 41-50

### Preparation of Rasagiline Tartrate Form I

General procedure: Rasagiline L-tartrate (150 mg) was heated in a solvent at reflux, and then allowed to cool to ambient temperature and stirred for 24 hours at this temperature before evaporation of the solvent. This procedure was used for the solvents indicated in Table 5 below.

Analytical data: XRD: Form I, see FIG. 21. IR: see FIG. 22.

TABLE 5

| | | | Aspect at | Aspect at | |
|---|---|---|---|---|---|
| Example | Solvent | Quantity | reflux | ambient | Result |
| 41 | acetone | 3 mL | suspension | suspension | Form I |
| 42 | chloroform | 3 mL | suspension | suspension | Form I |
| 43 | methanol | 0.5 mL | solution | suspension | Form I |
| 44 | MTBE | 3 mL | suspension | suspension | Form I |
| 45 | THF | 2 mL | solution | suspension | Form I |
| 46 | ethanol | 1 mL | solution | suspension | Form I |
| 47 | 2-butanone | 1.4 mL | solution | suspension | Form I |
| 48 | Methyl i- | 3 mL | suspension | suspension | Form I |
| | butylketone | | | | |
| 49 | water/ethanol | 0.5 mL | solution | suspension | Form I |
| | (20-80) | | | | |
| 50 | i-propyl acetate | 3 mL | suspension | suspension | Form I |

### Example 51

### Preparation of Rasagiline Mixture of Tartrate Form I and Hemitartrate Form I

Rasagiline base (1.61 g) was dissolved in 2-propanol (6.9 mL). L-Tartaric acid (1.41 g) was added and the mixture was stirred for 2 h at 40° C. The mixture was allowed to cool to ambient temperature and stirred for 24 hours at this temperature. The mixture was filtered and dried under vacuum at 40° C. Yield 2.04 g.

### Example 52

### Preparation of Rasagiline Hemitartrate Form I

Rasagiline base (150 mg) was dissolved in 2-propanol (1 mL). L-Tartaric acid (132 mg) was added and the mixture was stirred for 1 h at 40° C. The mixture was allowed to cool to ambient temperature and stirred for 24 hours at this temperature. The mixture was filtered and dried under ambient conditions.

Analytical data: XRD: Form I, see FIG. 23. IR: see FIG. 24.

### Example 53

### Preparation of Rasagiline Fumarate Form I

Rasagiline base (1.80 g) was dissolved in 2-propanol (7.7 mL). Fumaric acid (1.22 g) was added and the mixture was stirred for 2 h at 40° C. The mixture was allowed to cool to ambient temperature and stirred for 24 hours at this temperature. The mixture was filtered and dried under vacuum at 40° C. Yield 2.58 g.

Analytical data: XRD: Form I, see FIG. 25. IR: see FIG. 26.

### Examples 54-63

### Preparation of Rasagiline Fumarate Form I

General procedure: Rasagiline fumarate (150 mg) was heated in a solvent at reflux, and then allowed to cool to ambient temperature and stirred for 24 hours at this temperature before evaporation of the solvent. This procedure was used for the solvents indicated in Table 6 below.

TABLE 6

| | | | Aspect at | Aspect at | |
|---|---|---|---|---|---|
| Example | Solvent | Quantity | reflux | ambient | Result |
| 54 | acetone | 0.7 mL | solution | suspension | Form I |
| 55 | chloroform | 3 mL | suspension | suspension | Form I |
| 56 | methanol | 0.4 mL | solution | solution | Form I |
| 57 | MTBE | 1.4 mL | solution | suspension | Form I |
| 58 | THF | 0.7 mL | solution | suspension | Form I |
| 59 | ethanol | 0.7 mL | solution | suspension | Form I |
| 60 | 2-butanone | 0.8 mL | solution | suspension | Form I |
| 61 | Methyl i- | 2.5 mL | solution | suspension | Form I |
| | butylketone | | | | |
| 62 | water/ethanol | 0.5 mL | solution | suspension | Form I |
| | (20-80) | | | | |
| 63 | i-propyl acetate | 3 mL | suspension | suspension | Form I |

### Example 64

### Preparation of Rasagiline Hydrochloride Form I

Rasagiline base (2.49 g) was dissolved in 2-propanol (10.6 mL). Hydrochloric acid (1.43 g) was added and the mixture was stirred for 2 h at 40° C. The mixture was allowed to cool to ambient temperature and stirred for 24 hours at this temperature. The mixture was filtered and dried under vacuum at 40° C. Yield 0.37 g.

Analytical data: XRD: Form I, see FIG. 27. IR: see FIG. 28.

### Example 65

### Preparation of Rasagiline Hydrochloride Form II

Rasagiline base (15 g) was dissolved in 2-propanol (30 mL) at 40° C. Hydrochloric acid 37% (7 g) was added, and the mixture was stirred at 40° C. for 1 hour. The solid obtained was filtered, washed with 2-propanol, and dried at 40° C. under vacuum. 13.7 g of a white solid were collected (yield: 75.3%).

Analytical data: XRD: Form II, see FIG. 29. IR: see FIG. 30.

### Example 66

### Preparation of Rasagiline Besylate Form I

Rasagiline base (0.5 g) was dissolved in toluene (2 mL) at room temperature. Benzenesulfonic acid (0.46 g) was added at room temperature, and 1 mL more of toluene was added. At the beginning an oil was formed. The mixture was stirred for 3 h. The solid obtained was filtered and dried under vacuum at 40° C. 0.67 g of a white powder was collected (Yield: 69.8%).

Analytical data: XRD: Form I, see FIG. 31. IR: see FIG. 32.

### Example 67

### Preparation of Rasagiline Besylate Form I

Rasagiline base (10.05 g) was dissolved in toluene (40 mL) at room temperature. Benzenesulfonic acid (9.25 g) was added at room temperature. The mixture was stirred for 5 h. The solid obtained was filtered and dried under vacuum at 40° C. 19.27 g of a white powder was collected (Yield: 99.5%).

Analytical data: XRD: substantially identical to FIG. 31. IR: substantially identical to FIG. 32.

### Example 68

### Preparation of Rasagiline Besylate Form I

Rasagiline base (0.5 g) was dissolved in 2-propanol (2 mL) at room temperature. The solution obtained was light yellow. Benzenesulfonic acid (0.46 g) was added at room temperature. The solution was stirred for 3 h, but no precipitation was obtained. The solution was heated at 40° C. to allow evaporation. A white wax was obtained.

Analytical data: XRD: substantially identical to FIG. 31. IR: substantially identical to FIG. 32.

### Example 69

### Preparation of Rasagiline Mesylate

9.04 g of rasagiline mesylate and 36 mL of isopropanol were heated to reflux, until complete dissolution occurred, and stirred for 30 minutes at reflux. After this time, the mixture was cooled down to 0-5° C. and stirred for 30 minutes. The suspension was then filtered, and the collected solid was washed with 10 mL of isopropanol and dried at 50° C. for 4 h under vacuum. 8.66 g of white solid were thus obtained (95.80% yield).

### Example 70

### Flowability Studies of Rasagiline Salts

For samples of rasagiline salts, bulk and tapped densities were determined using a Jolting Volumeter Type STAV II apparatus from J. ENGELSMANN AG. The Hausner ratio of the rasagiline salt was calculated by dividing the tapped bulk density of the rasagiline salt by the bulk density of the rasagiline salt. Results are summarized in Table 7.

TABLE 7

| | Bulk | Tapped | | |
|---|---|---|---|---|
| Rasagiline | density | density | Hausner | |
| salt | (g/mL) | (g/mL) | ratio | Flowability |
| Mesylate | 0.236 | 0.407 | 1.72 | Very, very poor |
| Succinate | 0.492 | 0.513 | 1.04 | Excellent |
| Form I | | | | |
| Hydrochloride | 0.673 | 0.673 | 1.00 | Excellent |
| Form II | | | | |
| Hemitartrate | 0.138 | 0.181 | 1.31 | Passable |
| Form I | | | | |
| Besylate | 0.427 | 0.435 | 1.02 | Excellent |
| Form I | | | | |

### Example 71

### Solubility Studies in Water of Rasagiline Salts

The rasagiline salts were suspended in water under standard conditions (i.e. room temperature, normal pressure, ambient atmosphere), stirred until equilibration and filtered. The mother liquors were analyzed by HPLC. Results are summarized in Table 8.

TABLE 8

| Rasagiline salt | Solubility (g/L) | Descriptive term |
|---|---|---|
| Succinate Form I | >1200 | Very soluble |
| Hydrochloride Form II | About 400 | Freely soluble |
| Hemitartrate Form I | About 59 | Soluble |
| Besylate Form I | >800 | Freely soluble |

### Example 72

### Stability Studies of Rasagiline Salts

The rasagiline salts were stored under standard conditions (i.e. room temperature, normal pressure, ambient atmosphere). The samples were analyzed after 10 months by HPLC, XRD, and visual inspection. Results are summarized in Table 9.

TABLE 9

| | Purity (HPLC) | | XRD result | | Colour/Aspect |
|---|---|---|---|---|---|
| | | 10 months | | 10 months | 10 months |
| Rasagiline Salt | % initial | later | Initial | later | later |
| Mesylate | 99.58 | 99.8 | Form I | Form I | white powder |
| Benzoate | 44.75 | 50.29 | Form I | Form I | white-off |
| | | | | | powder |
| Galactarate | 98.92 | 98.81 | Form I + | Form I + | white-off |
| | | | galactaric | galactaric | powder |
| | | | acid | acid | |
| Gluconate | 98.61 | 96.36 | amorphous | amorphous | brown oil |
| D-Glucuronate | 86.00 | 50.64 | amorphous | amorphous | brown oil |
| Tosylate | 79.87 | 75.89 | Form I | Form I | white-off |
| | | | | | powder |
| Phosphate | 98.45 | 99.60 | amorphous | n.d.^{a} | white powder |
| Maleate | 66.54 | 63.27 | Form I | n.d.^{a} | white powder |
| Succinate | 99.29 | 99.61 | Form I | Form I | white powder |
| Acetate | n.d.^{a} | 98.01 | Form I | Form I | white-off |
| | | | | very low | powder |
| | | | | crystalline | |
| Acetate | 97.03 | 97.63 | Form II | Different | white-off |
| | | | | from Form | powder |
| | | | | I or II | |
| L-Tartrate | n.d.^{a} | 99.52 | Form I | Form I | white powder |
| L-Hemitartrate | 99.14 | 98.86 | Form I | Form I | white powder |
| Fumarate | 59.56 | 53.64 | Form I | n.d.^{a} | white-off |
| | | | | | powder |
| Hydrochloride | 99.18 | 99.74 | Form I | Form I | white powder |

^{a}Not determined.

### Example 73

### Accelerated Stability Studies of Rasagiline Salts

Rasagiline salts were submitted to accelerated conditions (40° C. and 75% RH) in two types of bags: polyethylene (PE) and aluminium (AL) bags. The samples were characterized by HPLC, XRD, and visual inspection. Results are summarized in Table 10.

TABLE 10

| | Purity (HPLC) | | | XRD | | | Aspect/Colour | | |
|---|---|---|---|---|---|---|---|---|---|
| | | 1st | 2nd | | 1st | 2nd | | 1st. | 2nd |
| Rasagiline Salt | % initial | Month | Month | Initial | Month | Month | Initial | Month | Month |
| Hydrochloride (PE) | >99.9% | >99.9% | >99.9% | Form II | Form II | Form II | white | white | white |
| Hydrochloride (AL) | | | | | | | powder | powder | powder |
| Succinate (PE) | >99.9% | >99.9% | 99.92% | Form I | Form I | Form I | white | white to | white to |
| Succinate (AL) | | | | | | | powder | yellow | yellow |
| | | | | | | | | powder | powder |
| Hemitartrate (PE) | 99.27% | 99.51% | 99.47% | Form I | Form I + | Form I + | white | white | white |
| | | | | | traces | traces | powder | powder | powder |
| Hemitartrate (AL) | | 99.49% | 99.55% | | tartrate | tartrate | | | |
| | | | | | Form I | Form I | | | |
| Besylate (PE) | >99.9% | >99.9% | n.d.^{a} | Form I | Form I + | n.d.^{a} | white | sticky | n.d.^{a} |
| | | | | | besylic | | powder | white | |
| | | | | | acid | | | solid | |
| Besylate (AL) | | | n.d.^{a} | | Form I | n.d.^{a} | | white | n.d.^{a} |
| | | | | | | | | powder | |

^{a}Not determined.

### Example 74

### Particle Size Studies of Rasagiline Salts

The particle size distribution of the rasagiline salts was determined. Results are summarized in Table 11.

TABLE 11

| Rasagiline salt | D(v, 0.1) | D(v, 0.5) | D(v, 0.9) |
|---|---|---|---|
| Succinate Form I | 23.6 μm | 85.1 μm | 180.4 μm |
| Hydrochloride Form II | 41.7 μm | 99.6 μm | 185.2 μm |
| Hemitartrate Form I | 4.5 μm | 18.3 μm | 78.8 μm |
| Besylate Form I | 6.5 μm | 28.8 μm | 50.6 μm |

Although the invention has been described and illustrated with a certain degree of particularity, it is understood that the disclosure has been made only by way of example, and that numerous changes in the conditions and order of steps can be resorted to by those skilled in the art without departing from the spirit and scope of the invention.

1. An acid addition salt of R-(+)-N-propargyl-1-aminoindan (i.e. rasagiline), 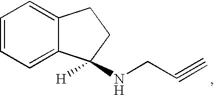
said addition salt of rasagiline having a Hausner ratio less than about 1.46.

2. The acid addition salt of rasagiline of claim 1, wherein the acid addition salt of rasagiline is in a crystalline form.

3. The acid addition salt of rasagiline of claim 2, wherein the acid is at least one of succinic acid, L-tartaric acid, hydrochloric acid, and benzenesulfonic acid.

4. The acid addition salt of rasagiline of claim 3, wherein said acid addition salt of rasagiline is rasagiline succinate Form I, wherein said rasagiline succinate Form I is characterized by an XRD pattern (2θ) (±0.2°) having characteristics peaks at approximately 10.1, 11.8, 13.5, 16.8, 17.9, 18.3, 18.6, 19.7, 19.9, 20.7, 21.3, 23.8, 24.2, 24.8, 26.5, 28.6 and 33.0°.

5. The rasagiline succinate Form I of claim 4, wherein said rasagiline succinate Form I is further characterized by an XRD pattern (2θ) (±0.2°) having additional characteristic peaks at approximately 9.5, 23.0, 25.7, 27.3 and 28.20.

6. A process for preparing the rasagiline succinate Form I of claim 4, said process comprising:
contacting rasagiline base with succinic acid, in the presence of a suitable solvent; and
removing the solvent.

7. The process of claim 6, wherein the solvent is a C₁-C₅ alcohol solvent.

8. A process for preparing the rasagiline succinate Form I of claim 4, said process comprising:
at least one of dissolving and slurrying rasagiline succinate in a suitable solvent; and
removing the solvent.

9. The process of claim 8, wherein the solvent is at least one of a ketone, a C₁-C₅ alcohol, an aliphatic ether, a C₁-C₅ ester, a halogenated aliphatic hydrocarbon, water and mixtures thereof.

10. The acid addition salt of rasagiline of claim 3, wherein said acid addition salt of rasagiline is rasagiline L-hemitartrate Form I, wherein said rasagiline L-hemitartrate Form I is characterized by an XRD pattern (2θ) (±0.2°) having characteristics peaks at approximately 6.6, 12.6, 16.5, 20.3, 22.9 and 23.0°.

11. The rasagiline L-hemitartrate Form I of claim 10, wherein said rasagiline L-hemitartrate Form I is further characterized by an XRD pattern (2θ) (±0.2°) having additional characteristic peaks at approximately 8.1, 13.2, 15.1, 17.3, 18.4, 19.5, 21.6, 21.7, 21.8, 22.2, 22.8, 24.3, 24.9, 26.7, 28.0, 29.6, 31.4, 32.5 and 36.5°.

12. A process for preparing the rasagiline L-hemitartrate Form I of claim 10, said process comprising:
contacting rasagiline base with a suitable amount of L-tartaric acid, in the presence of a suitable solvent; and
removing the solvent.

13. The process of claim 12, wherein the solvent is at least one of a C₁-C₅ alcohol, water and mixtures thereof.

14. The acid addition salt of rasagiline of claim 3, wherein said acid addition salt of rasagiline is rasagiline hydrochloride Form II, wherein said rasagiline hydrochloride Form II is characterized by an XRD pattern (2θ) (±0.2°) having characteristics peaks at approximately 8.9, 12.1, 14.4, 15.1, 17.2, 17.5, 21.1, 22.7, 23.1, 24.4, 25.1, 26.2, 26.4, 26.8, 27.9, 29.0, 32.0, 34.6, 36.5 and 38.9°.

15. A process for preparing the rasagiline hydrochloride Form II of claim 14, said process comprising:
contacting rasagiline base with hydrochloric acid, in the presence of a solvent comprising at least one C₁-C₅ alcohol and water, wherein the C₁-C₅ alcohol/water ratio (v/v) is less than or equal to 3; and
removing the solvent.

16. The process of claim 15, wherein the C₁-C₅ alcohol/water ratio (v/v) is equal to 3.

17. The acid addition salt of rasagiline of claim 3, wherein said acid addition salt of rasagiline is rasagiline besylate Form I, wherein said rasagiline besylate Form I is characterized by an XRD pattern (2θ) (±0.2°) having characteristics peaks at approximately 5.2, 10.4, 13.5, 14.2, 16.9, 18.1, 18.6, 19.5, 20.7, 22.2, 22.6, 23.8, 24.1, 25.3, 25.6, 26.2, 27.6, 28.7 and 29.5°.

18. A process for preparing the rasagiline besylate Form I of claim 17, said process comprising:
contacting rasagiline base with benzenesulfonic acid, in the presence of a suitable solvent; and
removing the solvent.

19. The process of claim 18, wherein the solvent is at least one of a C₁-C₅ alcohol, an aromatic hydrocarbon solvent, and mixtures thereof.

20. Use of an acid addition salt of rasagiline of claim 1 for preparing a pharmaceutical formulation.

21. Use of an acid addition salt of rasagiline of claim 1 for preparing rasagiline mesylate.
